(19) United States
(12) Patent Application Publication
(10) Pub. No.: US 2026/0115355 A1
(43) Pub. Date: Apr. 30, 2026

(54) BIOENGINEERING MUSCLE CONSTRUCTS USING 3D PRINTED MICRO- AND ULTRA-FINE FIBERS

(71) Applicant: University of Oregon, Eugene, OR (US)

(72) Inventors: Nick Willett, Eugene, OR (US); Robert E. Guldberg, Eugene, OR (US); Adam Rauff, Eugene, OR (US); Paul D. Dalton, Eugene, OR (US); Ievgenii Liashenko, Eugene, OR (US)

(73) Assignee: University of Oregon, Eugene, OR (US)

(21) Appl. No.: 19/368,808

(22) Filed: Oct. 24, 2025

Related U.S. Application Data
(60) Provisional application No. 63/711,562, filed on Oct. 24, 2024.
(60) Provisional application No. 63/876,774, filed on Sep. 5, 2025.

(51) Int. Cl. A61L 27/26; A61L 27/38; A61L 27/52; B33Y 10/00; B33Y 80/00; C08L 67/04; C08L 89/06; C12N 5/00

(52) U.S. Cl. CPC A61L 27/26; A61L 27/3804; A61L 27/52; B33Y 10/00; B33Y 80/00; C08L 67/04; C08L 89/06; C12N 5/0068; A61L 2430/30; C12N 2533/40; C12N 2533/54; C12N 2539/00

(57) ABSTRACT

Provided are ultrafine-fiber or microfiber scaffolds including a plurality of wall fibers and a plurality of reinforcing fibers, each reinforcing fiber including at least two (such as at least four) anchor segments and at least one (such as at least three) bridge segment, wherein: the wall fibers and the anchor segments are substantially aligned forming a plurality of walls, and the bridge segments each extend across the space between any two adjacent walls. Further provided are methods of producing an ultrafine-fiber or microfiber scaffold by electrohydrodynamic printing (EHD), including jetting along a first straight jetting path that is offset relative to the wall fiber direction at angle of less than 90°. Also provided are methods of culturing cells, forming tissues, and treating an injury using the disclosed scaffolds and compositions.

### CROSS REFERENCE TO RELATED APPLICATIONS

This application claims the benefit of U.S. Provisional Application No. 63/711,562, filed Oct. 24, 2024, and U.S. Provisional Application No. 63/876,774, filed Sep. 5, 2025, which are hereby incorporated by reference in their entirety.

### FIELD

This disclosure relates to tissue engineering, particularly by 3D printed microfiber constructs.

### BACKGROUND

Skeletal muscle is essential for locomotion, posture, respiration, and metabolism. Effective regeneration of skeletal muscles following injury is critical for maintaining health and quality of life. Impaired muscle function is classified as a disease and often leads to significant disability, chronic pain, and restricts an individual's ability to work and participate in recreational activities. Volumetric muscle loss (VML) injuries, resulting from blast trauma, car accidents, or tumor excision, often exceed innate muscle regeneration capabilities and result in diminished functional outcomes. To address such clinical challenges—primarily arising from aging, injury, or disease—there is a growing interest in regenerative therapies.

A major barrier to functional muscle regeneration is the disruption of the structural hierarchy that characterizes skeletal muscle tissue. Skeletal muscle features an organized, multi-scale architecture: at the micron scale sarcomeres form myofibrils; muscle fibers (individual muscle cells) are approximately 100 μm in diameter; and fascicles group these fibers into larger functional units (˜30 mm). This hierarchical organization is supported by connective tissue, including the endomysium around fibers, perimysium surrounding fascicles, and epimysium encompassing the entire muscle. The extracellular matrix (ECM) components bear a significant portion of passive loads, with the ECM modulus estimated to be 5-25 times greater than that of muscle cells. Crucially, these structures also facilitate lateral force transmission, enabling efficient mechanical load distribution across parallel aligned structures. The importance of preserving this architecture is underscored by the stark difference in healing outcomes between small and large muscle defects, as regeneration is significantly impaired when tissue loss exceeds a critical threshold. However, current engineered muscle constructs often lack sufficient size, structural alignment, and mechanical integrity to replicate these essential features.

Skeletal muscles experience diverse dynamic and passive loads and possess highly anisotropic mechanical properties, posing significant challenges for engineered constructs. Native muscle tissues are structurally adapted to withstand both active and passive forces, requiring robust mechanical support for stability. Nonetheless, current tissue-engineered muscle constructs fail to fully replicate this complex biomechanical environment. Many constructs exhibit poor architectural fidelity and substantial heterogeneity, particularly in thicker tissues. Engineered alignment features are typically 10- to 100-fold larger than the cellular scale, and achieving consistent alignment throughout large-volume constructs remains technically challenging. Moreover, 3D aligned constructs frequently suffer from insufficient mechanical stability, limiting their capacity to effectively transmit force longitudinally along myofibers and laterally through intramuscular connective tissue, both essential for muscle function.

Several tissue engineering strategies have attempted to address the need for alignment, but most fall short due to manufacturing limitations. Electrospun fiber membranes are widely used to produce aligned scaffolds with individual fiber diameters typically ranging from 150 nm-2 μm, with varying alignment and mechanical properties. However, their high fiber density restricts cell infiltration, typically limiting their application to two-dimensional formats. Additionally, the instability and whipping of the electrospun jet compromises precise control over scaffold architecture. Alternatively, sponge-like scaffolds enable control over porosity, including pore size, orientation, and interconnectivity, to enhance cell infiltration. Yet, increased porosity often reduces mechanical strength, and the achievable alignment remains insufficient for replicating native muscle architecture. Hydrogels delivered via bioprinting or injection methods provide benefits in terms of cell viability and spatial distribution but lack both structural strength and microscale alignment. Finally, soft lithography can produce micropatterned films with high fidelity surfaces cues; however, these cues are limited to surface interactions and lack the volumetric architectural control required for engineering thicker tissue constructs.

### SUMMARY

Provided herein are ultrafine-fiber or microfiber scaffolds including a plurality of wall fibers and a plurality of reinforcing fibers, each reinforcing fiber including at least two (such as at least four) anchor segments and at least one (such as at least three) bridge segment, wherein: the wall fibers and the anchor segments are substantially aligned forming a plurality of substantially parallel walls, and the bridge segments each extend across the space between any two adjacent walls from one anchor segment to another of the same reinforcing fiber, forming an angle of less than 90° with the two adjacent walls. In some aspects, the reinforcing fibers comprise at least one multi-bridge fiber which connects n adjacent walls with m bridge segments, wherein the m bridge segments are substantially parallel to each other, and n is any integer greater than 2 (such as greater than 3), and m=n−1. Also provided are compositions including the scaffold, which may further include a hydrogel and/or cells.

Further provided are methods of producing an ultrafine-fiber or microfiber scaffold including wall fibers and reinforcing fibers by electrohydrodynamic printing (EHD), the method including: (a) providing a first set of wall fibers that are substantially parallel to each other; and (b) EHD jetting along a first straight jetting path that is offset relative to the wall fiber direction at angle of less than 90°. Also provided are methods of culturing cells, forming tissues, and treating an injury using the disclosed scaffolds and compositions.

The foregoing and other features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1I: Representative light microscopy and scanning electron microscopy (SEM) images of three microfiber scaffold designs manufactured using melt-electrowriting (MEW), a 3D printing technique. FIGS. 1A-1C: Representative light microscopy images of “Isotropic” 5-fiber scaffold, “Aligned T” scaffold characterized by perpendicular running reinforcements, and “Aligned X” scaffold characterized by angled cross-bridge reinforcements. All scaffolds have circular shape with an overall diameter of 13.0 mm that fits in a 24-well plate. FIGS. 1D-1F: Representative SEM images of the Isotropic, Aligned T, and Aligned X scaffolds. The Isotropic scaffold has spacing between each parallel track of fibers of 150 μm, with fibers running at 0°, 36°, 72°, 108°, and 144°. The Aligned T scaffold architecture has spacing of 100 μm between aligned fiber walls and 1500 μm between reinforcing perpendicular walls. The Aligned X scaffold was designed with 100 μm between aligned fiber walls and 8 bridging fiber layers interlaced uniformly with the aligned layers. FIGS. 1G-1I: Representative higher magnification SEM images demonstrating the precision of the structural features manufactured by MEW on a small scale.

FIGS. 2A-2D: Model representation of Aligned X fiber architecture.

FIGS. 3A-3H: Representative images showing that two large (70-μm diameter) fibers reinforcing the scaffold along the perimeter improved its handleability. FIG. 3A: Aligned T scaffold without reinforcing rings wetted with ethanol and held with forceps. The scaffold contracted and folded to minimize the surface tension of the fluid. FIG. 3B: Isotropic scaffold without reinforcing rings wetted with ethanol and held with forceps folded in half. FIG. 3C: Aligned X scaffold with reinforcing rings (white arrow) placed next to aligned fibers without cross-bridge reinforcements or reinforcing rings. All scaffolds were wetted with ethanol, yet the non-reinforced scaffolds showed considerable deformation, whereas the reinforced scaffold maintained its shape. FIG. 3D: Aligned X scaffold with reinforcing rings wetted with ethanol and handled with forceps maintained its shape. FIGS. 3E-3G: SEM images showing reinforcing rings on the Isotropic (E), Aligned T (F), and Aligned X (G) scaffolds. FIG. 3H: Microfiber scaffolds with reinforcing rings printed on glass slides.

FIGS. 4A-4F: Representative micro-computed tomography (μCT) images of microfiber scaffold architectures demonstrate the cross-bridge reinforcement improved the structural stability of aligned fibers. All scaffolds were imaged after wetting and allowing for a minimum of 3 hours of drying. FIGS. 4A-4B: Top and isometric off-angled views of Isotropic scaffold. FIGS. 4C-4D: Top and isometric off-angled views of Aligned T scaffold. The aligned fiber tracks noticeably deformed and portrayed a wavy pattern away from the perpendicular reinforcements. FIGS. 4E-4F: Top and isometric off-angled views of Aligned X scaffold. The aligned fiber tracks in Aligned X demonstrated superior structural stability and integrity compared to Aligned T.

FIGS. 5A-5C: Tensile strength of dry, acellularized Isotropic, Aligned T, and Aligned X microfiber scaffolds. Tensile testing along the preferred fiber orientation revealed significant differences across scaffold designs. FIG. 5A: A plot comparing the Young's modulus of the three scaffold designs showing that Aligned X has the highest modulus that is more than double that of the Isotropic group. All three groups were significantly different from each other. FIG. 5B: A plot comparing the yield stress of the three scaffold designs showing that Isotropic requires significantly less load to reach plastic deformation. FIG. 5C: A plot comparing yield strain of the three scaffold designs showing significant differences across all groups, with Aligned T allowing for the most deformation prior to plastic deformation. The solid black line represents the mean of each distribution. Statistical significance was determined at p<0.05 (*). A one-way ANOVA was used with a Tukey's post-hoc.

FIG. 6: A plot showing that culturing cells on scaffolds did not influence their bulk mechanical properties. Scaffolds of Aligned X design were cultured with C2C12 myoblasts for 11 days with 40,000, 80,000, or 120,000 cells. Control groups included dry scaffolds that were never wetted or incubated, and acellular scaffolds incubated alongside the cellular scaffolds submerged in media. The groups did not include collagen hydrogel. After 11 days in culture, the constructs were allowed to dry for 1 hour and then tested under uniaxial tension. Prior to testing, each construct's mass was recorded and used for normalization in place of width and thickness. The slope of the force-length curve was recorded and normalized to the mass of each sample. The results showed that constructs softened when incubated in media, but the presence or number of cells did not significantly influence the stiffness.

FIGS. 7A-7K: Aligned microfiber architecture induced cellular alignment in composite constructs. FIGS. 7A-7C: Representative immunofluorescence microscopy images of composite muscle constructs cultured to Day 8, wherein the scaffold design is Isotropic (A), Aligned T (B), and Aligned X (C), respectively. Images were acquired with a 5× objective and stitched together to show the entire scaffold (scale bar: 3.0 mm). FIGS. 7D-7F: Representative individual 5× images demonstrating cellular organization on each scaffold design (scale bar: 500 μm). The Aligned T design had distinct cellular structures along the perpendicular reinforcements (E, white arrow). FIGS. 7G-7I: Representative higher magnification images acquired with a 20× objective (scale bar: 100 μm). The Aligned T design had distinct cellular structures along the perpendicular reinforcements, which impeded continuous cellular structures (H, white arrow). The DAPI channel was imaged using the 390 nm LED at 40% illumination with a 460 nm emission filter, while the F-actin channel was imaged using the 510 nm LED at 50% illumination with a 535 nm emission filter. All images were acquired with 100 ms exposure time. FIG. 7J: A plot comparing the uniaxial alignment index across composite constructs and three control groups including monolayers, hydrogel-only (Gel-only), and electrospun mesh (ES Mesh) without hydrogel. Composite constructs with the Aligned T and Aligned X scaffold designs had significantly higher alignment than the Isotropic constructs and the three control groups. FIG. 7K: A plot comparing the regional variance of mean direction amongst three technical replicates from different location in each sample. The Aligned T and Aligned X constructs had the lowest spatial variance, which was significantly lower than the monolayer and ES mesh groups. This demonstrates more spatial homogeneity in the aligned scaffold architectures. The solid black line represents the mean of each distribution. Statistical significance was determined at p<0.05 (*). A one-way ANOVA was used with a Tukey's post-hoc.

FIGS. 8A-8F: Representative immunofluorescence microscopy images overlayed on phase contrast images showing C2C12 myoblasts adhered to microfiber scaffolds of composite scaffold-hydrogel constructs cultured to day 8. The cellular cytoskeletons are in green, the nuclei in blue, and the microfiber scaffold in grayscale. FIGS. 8A-8B: Images of Isotropic composite constructs acquired with a 5× objective (A) and a 20× objective (B). FIGS. 8C-8D: Images of Aligned T composite constructs acquired with a 5× objective (C) and a 20× objective (D). FIGS. 8E-8F: Images of Aligned X composite constructs acquired with a 5× objective (E) and a 20× (F) objective.

FIGS. 9A-9F: Representative immunofluorescence microscopy images of C2C12 myoblasts cultured in monolayer (A-B), hydrogel only (C-D), and electrospun mesh (E-F), which serve as control for comparison with composite scaffold-hydrogel constructs.

FIGS. 10A-10I: Representative images showing that microfiber scaffolds provide reinforcement to composite constructs that prevents cell-mediated hydrogel contraction. FIGS. 10A-10H: Myoblasts cultured for 4 and 8 days on hydrogel only (A, E), and composite scaffold-hydrogel constructs with Isotropic (B, F), Aligned T (C, G), and Aligned X (D, H) scaffold designs. Images were acquired using a DSLR camera with the constructs in 24-well plates. The diameter of each well is 15.6 mm. FIG. 10I: A plot showing area retention at days 4 and 8 of each group. The hydrogel-only group (Gel-only in gray circles) had significantly less hydrogel area retained compared with composite constructs. The hydrogel-only group also had lower area retention on day 8 compared with day 4. The three composite constructs groups were not different from each other on day 4 or day 8. The Isotropic group had statistically decreased in hydrogel area retention from day 4 (87.0%) to day 8 (82.67%). The solid black line represents the mean of each distribution. Statistical significance was determined at p<0.05 (*). A two-way ANOVA was used with Sidak's multiple comparisons test.

FIGS. 11A-11D: Incorporation of a collagen hydrogel enhanced metabolic activity and improved cell seeding efficiency. FIG. 11A: A plot showing the results of alamarBlue assay of scaffold-only constructs and composite constructs 24 hours post seeding. Two different effective seeding densities of 400,000 and 800,000 cells/mL were studied. For the composite construct, the signal of the higher seeding density was stronger compared to that of the lower seeding density, while the scaffold-only group showed no difference between the seeding densities. FIG. 11B: A plot showing the results of picoGreen assay of the same groups in (A). Composite constructs had increased DNA mass with increased cell density, whereas the scaffold-only group showed no difference. The solid black line represents the mean of each distribution. Statistical significance was determined at p<0.05 (*). A two-way ANOVA was used with Sidak's multiple comparisons test. FIGS. 11C-11D: These plots include the results in (A-B) with the addition of control groups. Gel-only: hydrogel-only; monolayer: cell monolayer.

FIGS. 12A-12C: Incorporation of a collagen hydrogel enhanced cell retention and viability. FIG. 12A: A plot showing nuclear count for composite and scaffold-only constructs 24 hours post seeding, wherein the scaffolds are Isotropic, Aligned T, and Aligned X. A two-way ANOVA revealed that composite constructs had significantly higher number of nuclei compared with scaffold-only constructs (p<0.000001), while the effect of scaffold design was not significant (p=0.3876). FIG. 12B: A plot showing cellular viability for the same groups in (A). There was a significant difference between composite and scaffold-only constructs (p=0.0060), while the scaffold design did not influence viability (p=0.6154). FIG. 12C: A plot showing cellular proliferation for the same groups in (A). There was increased proliferation in composite constructs compared with scaffold-only constructs (p=0.0230), and scaffold design influenced proliferation (p=0.0429). The solid black line represents the mean of each distribution. Statistical significance was determined at p<0.05 (*). A two-way ANOVA was used with Sidak's multiple comparisons test.

FIGS. 13A-13J: Composite constructs with Aligned X design had increased myotube formation. FIGS. 13A-13F: Representative immunofluorescence microscopy images of composite constructs with Isotropic (A, D), Aligned T (B, E), and Aligned X (C, F) microfiber scaffold designs. (A-C) show three colors with blue representing nuclei, green representing myosin heavy chain (MHC), and red representing F-actin. (E-F) only show the green channel representing MHC. The acquisition settings included laser power of 3.0%, 2.4%, and 2.0% for the blue, green, and red excitation lasers, a gain of 849,578, 617.208, and 759.740 for the blue, green, and red emission channels, and a pixel dwell time of 2.576·10⁻⁷ seconds. FIG. 13G: A plot comparing the area fraction of myotubes across the three groups showing that the Aligned X group had the highest myotube area fraction that was statistically different from the Isotropic group. FIG. 13H: A plot comparing the mean fluorescence intensity of MHC across the three groups, showing no statistical difference. FIG. 13I: A plot comparing myotube diameter across the three groups showing that the Aligned X group had significantly larger myotube diameters than the Isotropic and Aligned T groups. FIG. 13J: A plot comparing the number of myotubes across the three groups showing that the myotube count in the Aligned T and Aligned X groups was significantly higher than in the Isotropic group. The solid black line represents the mean of each distribution. Statistical significance was determined at p<0.05 (*). A one-way ANOVA was used with a Tukey's post-hoc test.

FIGS. 14A-14D: Aligned microfiber scaffolds without reinforcements had considerable deformations. FIGS. 14A-14B: Representative images showing that aligned microfibers wetted with ethanol in a petri dish exhibited significant shape changes (A), and further deformed when handled with forceps (B). FIGS. 14C-14D: Representative 20× phase contrast and immunofluorescence microscopy images of composite constructs with aligned microfibers without reinforcements, showing considerable dimensional instability (scale bar: 500 μm).

FIGS. 15A-15C: Schematics showing scaffolds with bridging reinforcing fibers generated by EHD utilizing printing paths with different offset angles. FIG. 15A: A reinforcing multi-bridge fiber shown in red deposited on top of aligned fiber walls (gray) using a programmed printing path (dashed black line). FIG. 15B: 3 printing paths at offset angles to the aligned fibers of 3°, 10°, and 25° demonstrating the relation of the offset angle of a printing path and the frequency of bridge segments. This shows a higher offset angle results in more fiber bridges. FIG. 15C: A bridge segment of a reinforcing fiber is the segment that is suspended across one aligned fiber wall to its adjacent aligned fiber wall. The anchor segment of a reinforcing fiber is the segment that runs along the fiber wall. The ratio of the length of a bridge segment to the length of an anchor segment is a function of the angle of the printing path and the aligned fiber walls. The reinforcing fibers are not straight segments due to the electrostatic autofocusing effect observed in electrostatic additive manufacturing. The printing path that induces bridging fibers may be non-linear, and spacing between those paths can be variable to induce spatially varying constructs with gradients of density and mechanical properties. This method enables controllable introduction of bridge segments by programming their frequency and location via: i) printing path angle, ii) proportion of reinforcing fibers to the wall fibers, or 3) location of the reinforcing fibers through scaffold height (layer number where reinforcements are introduced).

FIG. 16: A schematic showing a top view of a scaffold utilizing repeating multi-bridge fibers and corresponding mirroring fibers.

FIG. 17: A schematic showing generation of a reinforcing layer during EHD printing. The deposited layer is highlighted as it is extruded from a nozzle and forms a stable jet. The number of bridge segments matches the number of times the printing path crosses aligned walls.

FIGS. 18A-18E: Schematics showing reinforcing fiber layers in microfiber scaffolds. FIG. 18A: An isometric view of an exemplary microfiber scaffold with one reinforcing fiber anchored into multiple aligned fiber walls highlighted. FIG. 18B: A side view showing reinforcing fibers at regularly spaced layers across the thickness of a scaffold. FIG. 18C: A top view of an exemplary microfiber scaffold with reinforcing fibers highlighted. FIG. 18D: An isometric view showing 8 layers of reinforcing fibers along the thickness of the aligned fiber walls. FIG. 18E: One aligned fiber wall with 8 reinforcing fiber layers highlighted.

FIG. 19: An image showing an example of suturing of a scaffold to a tibialis anterior muscle by attaching the four corners of the scaffold with standard PDO sutures.

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided.

About: Unless context indicates otherwise, “about” refers to plus or minus 5% of a reference value. For example, “about” 100 refers to 95 to 105.

Administration: The introduction of a substance or composition into a subject by a chosen route. Administration can be local or systemic. In some examples, administration includes placement or implantation of a composition (e.g., a scaffold, a scaffold-hydrogel composite material, etc.) to a target site within a subject (such as an injury site of the musculoskeletal system), e.g., by any suitable surgical, or minimally invasive procedures.

Anisotropic: Having or exhibiting a physical property with different values when measured in different directions. Structural anisotropy of tissues in vivo arises from the preferred orientation of extracellular matrix proteins and/or cells, which imparts tissues with designated functions. Anisotropy can be quantified by measuring the mechanical characteristics of materials, or by image analysis, x-ray scattering, or mass transport characterization. In some examples, mechanical characterization involves measurements of direction-specific (e.g., longitudinal vs. transverse) elastic moduli.

Biocompatible: The property of not causing undesirable local or systemic effects in a subject when administered at an amount effective for its purpose. In some examples, a biocompatible material (e.g., polymer) is melted to form fibers in EHD or MEW. Examples of biocompatible materials that can be used in accordance with the present disclosure include synthetic polymers (e.g., poly(α-hydroxy acids), such as poly(lactic acid) (PLA), poly(lactic-co-glycolic acid) (PLGA), and poly(glycolic acid) (PGA); poly(ε-caprolactone) (PCL); polydioxanone (PDO); poly(vinyl alcohol) (PVA); poly(L-lactide-co-ε-caprolactone) (PLCL); polyurethanes (PU); nylon (polyamide); thermoplastic elastomers (TPEs); liquid crystal elastomers (LCEs); polyether ether ketone (PEEK); polyether ketone ketone (PEKK); poly(orthoesters); polycarboxymethylcellulose (CMC); polyvinylpyrrolidone (PVP); poly(vinylalcohol) (PVA); and polyethylene glycol (PEG)), natural polymers (e.g., sugars, such as trehalose, sucrose, glucose, lactose, and maltodextrin; dextrans; gelatin; collagen; silk; chitosan; poly-γ-glutamate; hyaluronic acid (HA); and methacrylated hyaluronic acid (MeHA)), or any combination thereof. In some examples, the biocompatible material is thermoplastic.

Electrohydrodynamic printing (EHD): A class of additive manufacturing technologies in which an electric field is used to control the ejection and deposition of a material. Melt electrowriting (MEW) is a type of EHD in which a molten material (e.g., polymer) is jetted and directed by the electric field.

Fiber: An elongated, filament-like structure having a length substantially greater than its cross-sectional dimensions. In EHD, fibers are formed by applying an electric field to draw a viscoelastic or molten material from a nozzle into a fine jet, which solidifies or stabilizes as it travels toward a collector, forming a fiber. “Reinforcing fiber” refers to a fiber including at least two anchor segments and at least one bridge segment, wherein the two anchor segments form parts of two separate walls, and the bridge segment extends across the walls from one anchor segment to the other. “Wall fiber” refers to fibers that are used to form walls that are not reinforcing fibers.

Gel: A colloidal system comprising a solid three-dimensional network within a liquid. By weight, a gel is primarily liquid, but behaves like a solid due to a three-dimensional network of entangled and/or crosslinked molecules of a solid within the liquid. From a rheological perspective, a gel has a storage modulus G′ value which exceeds that of the loss modulus G″. The storage modulus is a measure of the energy stored in a material in which a deformation (e.g., sinusoidal oscillatory shear) has been imposed; storage modulus can be thought of as the proportion of total rigidity of a material that is attributable to elastic deformation. The loss modulus is a measure of the energy dissipated in a material in which a deformation (e.g., sinusoidal oscillatory shear) has been imposed; loss modulus can be thought of as the proportion of the total rigidity of a material that is attributable to viscous flow rather than elastic deformation. The storage modulus and loss modulus can be determined with a rheometer.

Hydrogel: A three-dimensional crosslinked hydrophilic polymer. Hydrogels include a mixture of porous, permeable polymers and at least 10% by weight or volume of interstitial fluid (e.g., water). They can be highly absorbent yet maintain well defined structures. Hydrogels can be prepared using natural or synthetic polymeric materials, including hyaluronic acid, poly(ethylene glycol), collagen (e.g., type I collagen), alginate, agarose, chitosan, heparin, fibrin, gelatin methacrylate, gelatin, and Matrigel (solubilized basement membrane matrix secreted by Engelbreth-Holm-Swarm (EHS) mouse sarcoma cells). Hydrogels may be combined with one or more proteins, such as laminin and fibronectin, to form a heterogeneous material composition. In some examples, hydrogels enclose or embed cells.

Isolated or purified: An isolated or purified biological substance (such as a nucleic acid, protein, protein complex, or cell) has been substantially separated, or produced apart from other biological substances, with which it is naturally associated. An isolated or purified biological substance can be obtained through isolation or purification from samples obtained from an organism; recombinant expression or production in host cells followed by purification, and chemical synthesis followed by purification. The term “isolated” or “purified” does not require absolute purity; rather, it is intended as a relative term, for example, referring to being more enriched with (or having a higher concentration of) the substance, compared to a crude preparation or a natural environment from which the substance is isolated or purified. In some aspects, a biological substance is purified if the substance represents at least 50%, at least 60%, at least 70%, at least 80%, at least 90%, at least 95%, at least 96%, at least 97%, at least 98%, at least 99%, at least 99.5%, or greater, of the total moles of non-solvent substances in a preparation.

Layer: Referring to a pattern of fibers, designed to be deposited onto a substrate (e.g., a collector) within one defined stage of a printing process (e.g., EHD or MEW), before the next stage of deposition begins. For example, each layer may be designed to be printed at a specific angle relative to a reference line (e.g., relative to fibers of the first layer). For instance, the angle of the printing path for different layers may be: layer n: 0°, layer n+1: ±3°, layer n+2: 0° . . . ; or layer n: 0°, layer n+1: 0°, layer n+2: ±3° . . . ; or layer n: 0°, layer n+1: ±3°, layer n+2: ±3° . . . , wherein ± refers to + or −, e.g., layer n+1: +3°, layer n+2: −3°. Layers are defined with respect to designs or instructions given to a printing system (e.g., EHD or MEW). Thus, the fibers constituting a layer may or may not eventually be presented in the same structural layer of the printed product, as fibers may sink or distort during the printing process. A reinforcing layer refers to a layer that includes at least one reinforcing fiber (i.e., a continuous fiber that includes at least two anchor segments and at least one bridge segment). A wall layer refers to a layer that does not include a reinforcing fiber.

Microfiber and ultrafine-fiber: “Microfiber” refers to a fiber having a diameter of less than about 100 μm. “Ultrafine fiber” refers to a fiber having a diameter of less than about 1 μm. Thus, “ultrafine-fiber” is encompassed by “microfiber” as used herein.

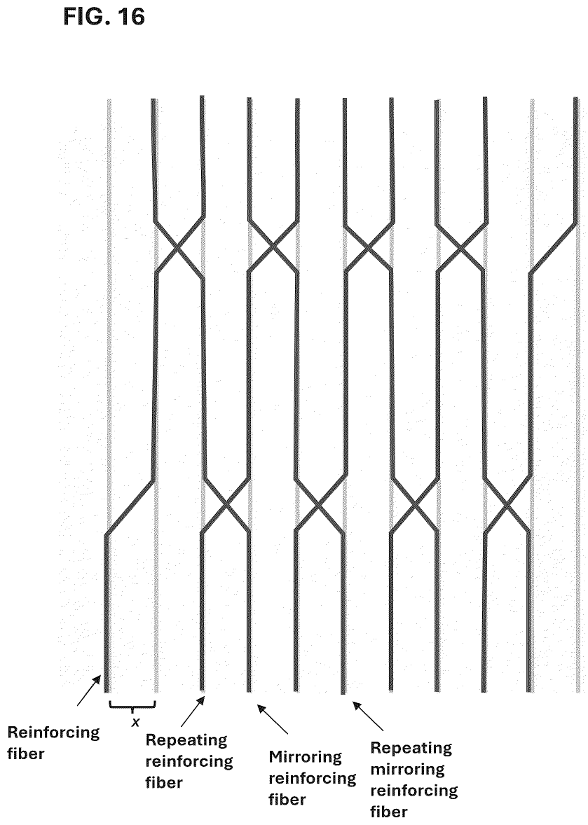

Mirroring: Referring to two elements mirroring the features (e.g., that are geometric reflections) of each other with respect to a reference plane or axis. See, e.g., the reinforcing fiber and the mirroring reinforcing fiber shown in FIG. 16.

Musculoskeletal tissue: Tissues that make up the musculoskeletal system, which provides support, stability, movement, and protection to the body, including muscle tissues, skeletal tissues (bones and cartilage), connective tissues (ligaments, tendons, and fascia), and joints. Cells from musculoskeletal tissues are cells that found in these tissues, which may include tenocytes, ligamentocytes, osteoblasts, osteocytes, osteoclasts, bone lining cells, chondroblasts, chondrocytes, progenitor cells, skeletal muscle fibers, satellite cells, myoblasts, myosatellite cells,, fibroadipogenic progenitors, myofibroblasts, type A synoviocytes, type B synoviocytes, fibroblasts, mesenchymal stem cells, endothelial cells, pericytes, macrophages, etc.

Scaffold: A three-dimensional structure formed from fibers (e.g., by EHD printing), configured to provide mechanical support or a framework for the attachment, organization, alignment, or growth of other materials (e.g., hydrogel, and/or cells).

Spacing: Referring to the smallest distance between two corresponding structural elements (e.g., walls, bridge segments, anchor segments, etc.), wherein “distance” refers to the length of a line connecting two points respectively located on the different elements.

Subject: Living multi-cellular vertebrate organisms, a category that includes human and non-human mammals, such as non-human primates, rats, mice, dogs, cats, horses, sheep, cows, rabbits, and pigs. In some examples, a subject is a human. In some examples, a subject is a veterinary subject. In some examples, the subject has a musculoskeletal injury, including damages to skeletal muscles, bones, tendons, joints, ligaments, and other affected soft tissues. In some examples, the subject has a skeletal muscle injury, volumetric muscle loss (VML), a tendon injury (such as a tendon rupture, tendon laceration, or tendinosis), a bone injury (such as a fracture, such as a non-union fracture, or due to cancer, osteoporosis, or osteoarthritis), or a wound (a damage or injury to living tissue).

Substantially aligned: “Substantially aligned” refers to fibers oriented in generally the same direction and deposited in contact or close proximity to one another, such that as a group they define a wall-like structure that may be vertical or inclined relative to a reference plane. The term encompasses fibers that are perfectly aligned as well as those exhibiting (or having a portion exhibiting) minor angular deviations from one another (for example, within about ±10°, ±5°, ±2°, or ±1°), provided that their collective orientation maintains the overall wall-like configuration. “Aligned” is used to describe fibers stacked or positioned along their lengths such that their longitudinal axes are parallel, and as a group, define a wall-like structure that may be vertical or inclined relative to a reference plane. “Aligned” is encompassed by “substantially aligned” as used herein.

Substantially parallel: “Substantially parallel” is used to describe two or more structural elements (e.g., walls, bridge segments, etc.) that extend in generally the same direction such that any deviation from exact parallelism is no more than about ±10°, such as within about ±5°, such as within about ±2°. “Parallel” is used to describe both exactly parallel elements (0° deviation) and elements that deviate from exact parallelism by no more than about ±1°. Such minor deviations are considered within the scope of “parallel” to account for manufacturing tolerances. “Parallel” is encompassed by “substantially parallel” as used herein.

Wall: A structure formed by stacking fibers. For example, a wall may include entire continuous fibers (wall fibers), and optionally, fiber segments (anchor segments), that are stacked or positioned with adjacent fibers or fiber segments in a substantially aligned manner. The wall may be vertical or inclined relative to a reference plane (e.g., a horizonal plane, or a base plane on which the wall is formed or supported). In some examples, the wall may also include regions containing holes or interstitial spaces, where a subsequently deposited fiber does not fully conform to the contour of one or more underlying fibers. In such regions, a wall fiber may have one or more suspended portions, making the wall perfused.

### II. Overview

Effective regeneration of skeletal muscle with highly aligned fiber architecture remains a significant challenge in tissue engineering. Structural alignment of muscle constructs along with mechanical integrity are crucial for effective engineering of grafts and microphysiological systems. As demonstrated herein, an emerging 3D printing technology called melt electrowriting (MEW) enables the fabrication of microfiber scaffolds with controlled porosity, mechanical integrity, and architectural design over multiple scales. With the methods and systems disclosed herein, MEW allows for the creation of highly anisotropic structures spanning the cellular and tissue scales and supports the manufacturing of large tissue constructs with high porosity.

Scaffold designs available in the art prior to the present application are susceptible to deformation, necessitating periodic reinforcement with larger fibers or other structures. Although reinforcement structures help maintain mechanical stability, their incorporation reduces scaffold porosity and can negatively affect cellular organization. Conversely, removing such reinforcements significantly compromises structural integrity, posing a persistent limitation for tissue-engineered muscle constructs.

Provided herein are novel scaffolds, composite materials (e.g., composites of scaffolds and hydrogels, such as type I collagen hydrogels), methods of manufacturing scaffolds and materials by MEW, and methods of cell culturing, tissue engineering, and treatment. The scaffolds, materials, and methods provided herein enable the fabrication of large-scale, highly aligned, and mechanically stable tissue constructs (e.g., muscle), which demonstrate improved overall structural integrity, increased cell fusion and differentiation, highly aligned cellular organization, and enhanced myogenic maturation and differentiation.

Large and highly aligned, engineered tissue constructs of skeletal muscle were developed using a hydrogel and a microfiber scaffold. The approach enables production of large and scalable tissue constructs with robust mechanical integrity that resists soft tissue contraction, a challenge in tissue engineering. The novel microfiber scaffold architecture provided structural integrity and geometrical continuity of aligned fiber walls compared with other designs previously employed. The composite system enabled formation of highly aligned and multinucleated myotubes while maintaining bulk construct shape by resisting cell-mediated contractions. The novel scaffold architecture significantly improved muscle cell differentiation and maturation.

### III. Scaffolds

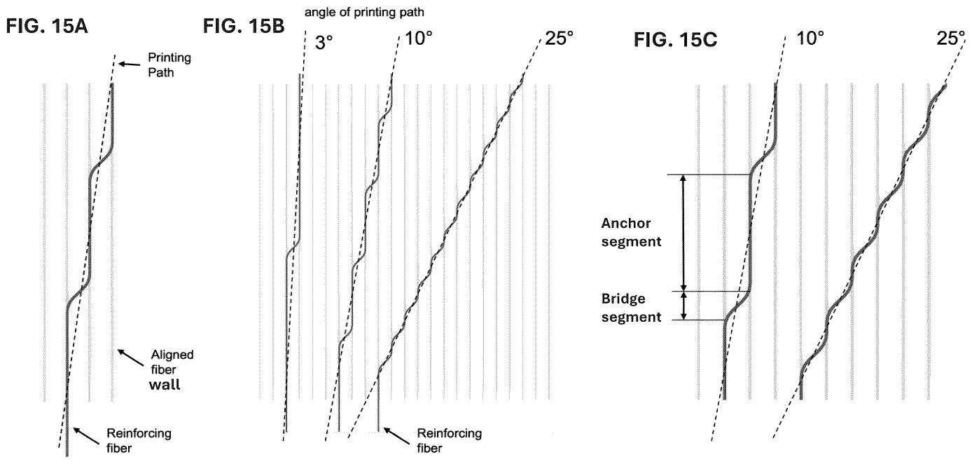

Provided herein are ultrafine-fiber or microfiber scaffolds including a plurality of wall fibers and a plurality of reinforcing fibers, wherein each reinforcing fiber includes at least two anchor segments and at least one bridge segment, wherein: the wall fibers and the anchor segments are substantially aligned forming a plurality of substantially parallel walls, and the bridge segments each extend across the space between any two adjacent walls from one anchor segment to another of the same reinforcing fiber, forming an angle of less than 90° with each of the two adjacent walls (e.g., FIG. 15A). A reinforcing fiber that includes more than one bridge segment is referred to as a multi-bridge fiber.

In some aspects, the scaffolds include repeating reinforcing fibers, with the spacing between two adjacent repeating reinforcing fibers being about x to about (l−m−1)x, wherein l is the number of walls included in the scaffold (which may be any integer greater than 3, such as less than 1,000,000, such as less than 100,000, such as about 10 to about 10,000, such as about 10 to about 5,000, such as about 10 to about 1000, such as about 50 to about 150, or about 50, 100, 500, 1,000, or 5,000, etc.), and m is the number of bridge segments that a reinforcing fiber has, and x is the spacing between two adjacent walls (e.g., FIG. 16).

In some aspects, the scaffolds include pairs of mirroring reinforcing fibers. In some examples, the scaffold includes, for one or more (such as at least 10%, 20%, 30%, 40%, 50%, 60%, 65%, 70%, 75%, 80%, 85%, 90%, 95%, 96%, 97%, 98%, or 99%, or all) of the repeating reinforcing fibers, a mirroring reinforcing fiber, wherein a bridge segment of the reinforcing fiber and a bridge segment of the corresponding mirroring reinforcing fiber pass each other (with or without touching). As used herein, reinforcing fibers include mirroring reinforcing fibers, if present, unless the context indicates otherwise.

In some aspects, for scaffolds that do not have mirroring reinforcing fibers, they have a property of being “collapsable,” where each bridge segment is rotatable as a hinge, and thus can minimize the spacing between walls. This type of scaffold can be effectively sheared, with its width significantly decreased. This property is conducive to delivering the scaffold through a fine needle or incision (e.g., during laparoscopic procedures) and subsequently allowing it to recover its original shape within the subject.

In some aspects, each of the wall fibers and reinforcing fibers have a diameter of less than about 100 μm (such as less than about 90 μm, about 80 μm, about 70 μm, about 65 μm, about 60 μm, about 55 μm, about 50 μm, about 45 μm, about 40 μm, about 35 μm, about 30 μm, about 25 μm, about 20 μm, about 15 μm, about 14 μm, about 13 μm, about 12 μm, or about 11 μm), or more than about 0.1 μm (such as more than about 0.2 μm, about 0.3 μm, about 0.4 μm, about 0.5 μm, about 0.6 μm, about 0.7 μm, about 0.8 μm about 0.9 μm, about 1 μm, about 1.5 μm, about 2 μm, about 2.5 μm, about 3 μm, about 3.5 μm, about 4μm, about 4.5 μm, or about 5 μm), or both (such as about 0.1 μm to about 100 μm, about 0.1 μm to about 50 μm, about 0.1 μm to about 40 μm, about 0.1 μm to about 30 μm, about 0.1 μm to about 20 μm, about 0.1 μm to about 15 μm, about 0.1 μm to about 12 μm, about 0.1 μm to about 10 μm, about 0.5 μm to about 100 μm, about 0.5 μm to about 50 μm, about 0.5 μm to about 40 μm, about 0.5 μm to about 30 μm, about 0.5 μm to about 20 μm, about 0.5 μm to about 15 μm, about 0.5 μm to about 12 μm, about 0.5 μm to about 10 μm, about 1 μm to about 100 μm, about 1 μm to about 50 μm, about 1 μm to about 40 μm, about 1 μm to about 30 μm, about 1 μm to about 20 μm, about 1 μm to about 15 μm, about 1 μm to about 12 μm, or about 1 μm to about 10 μm). In some examples, the diameter of each of the wall fibers and reinforcing fibers is about the same.

In some aspects, the angle formed between the bridge segment and the walls is greater than 3°. In some examples, the angle is about 10° to about 85°, about 10° to about 70°, about 10° to about 65°, about 10° to about 60°, about 10° to about 55°, about 10° to about 50°, about 20° to about 50°, about 25° to about 50°, about 10° to about 45°, about 20° to about 45°, about 25° to about 45°, about 10° to about 40°, about 20° to about 40°, about 25° to about 40°, about 10° to about 35°, about 20° to about 35°, about 25° to about 35°, about 10° to about 30°, about 20° to about 30°, about 25° to about 30°, or about 26° to about 27°.

In some aspects, the reinforcing fibers include at least one (such as at least 10%, 20%, 30%, 40%, 50%, 60%, 65%, 70%, 75%, 80%, 85%, 90%, 95%, 96%, 97%, 98%, or 99%, or all of total reinforcing fibers) multi-bridge fiber which connects n adjacent walls with m bridge segments, wherein the m bridge segments are substantially parallel to each other, and n is any integer greater than 2 (such as 3, 4, 5, 6, 7, 8, 9, 10 . . . or higher, up to the total number of walls included in the scaffold), and m=n−1. In some examples, n is any integer greater than 3 (such as 4, 5, 6, 7, 8, 9, 10 . . . or higher, up to the total number of walls included in the scaffold, such as about 4 to about 50, about 4 to 40, about 4 to about 30, about 4 to about 25, about 4 to about 20, about 4 to about 15, about 4 to about 10, etc.). In some examples, at least 50%, 60%, 65%, 70%, 75%, 80%, 85%, 90%, 95%, 96%, 97%, 98%, or 99%, or all of the multi-bridge fibers have the same n and m. In some examples, the angle formed by each of the m bridge segments and the walls it bridges is at least 3°. In some examples, the angle is about 10° to about 85°, about 10° to about 70°, about 10° to about 65°, about 10° to about 60°, about 10° to about 55°, about 10° to about 50°, about 20° to about 50°, about 25° to about 50°, about 10° to about 45°, about 20° to about 45°, about 25° to about 45°, about 10° to about 40°, about 20° to about 40°, about 25° to about 40°, about 10° to about 35°, about 20° to about 35°, about 25° to about 35°, about 10° to about 30°, about 20° to about 30°, about 25° to about 30°, or about 26° to about 27°.

In some aspects, the wall fibers constitute about 50.0% to about 99.9% by weight of the microfiber scaffold (such as about 50% to about 99%, about 55% to about 99%, about 60% to about 99%, about 65% to about 99% about 70% to about 99%, about 75% to about 99%, about 80% to about 99%, about 85% to about 99%, about 90% to about 99%, about 50% to about 95%, about 55% to about 95%, about 60% to about 95%, about 65% to about 95% about 70% to about 95%, about 75% to about 95%, about 80% to about 95%, or about 85% to about 95%), and/or the reinforcing fibers constitute about 0.1% to about 50.0% by weight of the microfiber scaffold (such as about 1% to about 50%, about 1% to about 45%, about 1% to about 40%, about 1% to about 35% about 1% to about 30%, about 1% to about 25%, about 1% to about 20%, about 1% to about 15%, about 1% to about 10%, about 5% to about 50%, about 5% to about 45%, about 5% to about 40%, about 5% to about 35% about 5% to about 30%, about 5% to about 25%, about 5% to about 20%, or about 5% to about 15%). In some examples, the scaffold includes only wall fibers and reinforcing fibers.

In some aspects, the ratio of the number of reinforcing fibers to the number of the wall fibers is about 1:1,000,000 to about 1:1 (reinforcing fibers:wall fibers) (such as about 1:100,000 to about 1:10, about 1:10,000 to about 1:10, about 1:1,000 to about 1:10, about 1:500 to about 1:10, about 1:400 to about 1:10, about 1:300 to about 1:10, about 1:250 to about 1:10, about 1:200 to about 1:10, about 1:150 to about 1:10, about 1:100 to about 1:10, about 1:90 to about 1:10, about 1:80 to about 1:10, about 1:70 to about 1:10, about 1:60 to about 1:10, about 1:50 to about 1:10, about 1:40 to about 1:10, about 1:30 to about 1:10, about 1:20 to about 1:10, about 1:50 to about 1:5, about 1:40 to about 1:5, about 1:30 to about 1:5, about 1:20 to about 1:5, about 1:15 to about 1:5, or about 1:5 to about 1:1).

In some aspects, the ratio of the number of reinforcing layers to the number of wall layers is about 1:1,000 to about 1:1 (reinforcing layers:wall layers), such as about 1:1,000 to about 1:3, about 1:1,000 to about 1:5, about 1:1,000 to about 1:8, about 1:1,000 to about 1:10, about 1:500 to about 1:3, about 1:500 to about 1:5, about 1:500 to about 1:8, about 1:500 to about 1:10, about 1:400 to about 1:3, about 1:400 to about 1:5, about 1:400 to about 1:8, about 1:400 to about 1:10, about 1:300 to about 1:3, about 1:300 to about 1:5, about 1:300 to about 1:8, about 1:300 to about 1:10, about 1:200 to about 1:3, about 1:200 to about 1:5, about 1:200 to about 1:8, about 1:200 to about 1:10, about 1:100 to about 1:3, about 1:100 to about 1:5, about 1:100 to about 1:8, about 1:100 to about 1:10, about 1:50 to about 1:3, about 1:50 to about 1:5, about 1:50 to about 1:8, about 1:50 to about 1:10, about 1:30 to about 1:3, about 1:30 to about 1:5, about 1:30 to about 1:8, about 1:30 to about 1:10, about 1:5 to about 1:1, about 2:9 to about 1:1, about 1:4 to about 1:1, about 2:7 to about 1:1, about 1:3 to about 1:1, about 2:5 to about 1:1, or about 2:5. In some examples, the reinforcing layers are regularly spaced in the scaffold. For instance, a reinforcing layer is introduced for every 1 to up to 1/2 of the total number of wall layers (such as 1, 2, 3, 4, 5 . . . 100 . . . 1000 . . . ) wall layers. In some examples, the scaffold includes only reinforcing layers and wall layers.

In some aspects, the spacing between any two adjacent walls is about 40 μm to about 500 μm, such as about 40 μm to about 400 μm, about 40 μm to about 350 μm, about 40 μm to about 300 μm, about 40 μm to about 250 μm, about 40 μm to about 200 μm, about 40 μm to about 150 μm, about 50 μm to about 500 μm, about 50 μm to about 400 μm, about 50 μm to about 350 μm, about 50 μm to about 300 μm, about 50 μm to about 250 μm, about 50 μm to about 200 μm, about 50 μm to about 150 μm, about 60 μm to about 500 μm, about 60 μm to about 400 μm, about 60 μm to about 350 μm, about 60 μm to about 300 μm, about 60 μm to about 250 μm, about 60 μm to about 200 μm, about 60 μm to about 150 μm, about 65 μm to about 500 μm, about 65 μm to about 400 μm, about 65 μm to about 350 μm, about 65 μm to about 300 μm, about 65 μm to about 250 μm, about 65 μm to about 200 μm, about 65 μm to about 150 μm, about 70 μm to about 500 μm, about 70 μm to about 400 μm, about 70 μm to about 350 μm, about 70 μm to about 300 μm, about 70 μm to about 250 μm, about 70 μm to about 200 μm, about 70 μm to about 150 μm, about 75 μm to about 500 μm, about 75 μm to about 400 μm, about 75 μm to about 350 μm, about 75 μm to about 300 μm, about 75 μm to about 250 μm, about 75 μm to about 200 μm, about 75 μm to about 150 μm, about 80 μm to about 500 μm, about 80 μm to about 400 μm, about 80 μm to about 350 μm, about 80 μm to about 300 μm, about 80 μm to about 250 μm, about 80 μm to about 200 μm, about 80 μm to about 150 μm, about 85 μm to about 500 μm, about 85 μm to about 400 μm, about 85 μm to about 350 μm, about 85 μm to about 300 μm, about 85 μm to about 250 μm, about 85 μm to about 200 μm, about 85 μm to about 150 μm, or about 100 μm. Spacing refers to the smallest distance between two walls. Thus, both parallel walls, and non-parallel walls that do not intersect in the scaffold have one spacing value. In some examples, the spacing between any two adjacent walls is the same.

In some aspects, the scaffold includes l walls, and includes a plurality of repeating multi-bridge fibers each connects n adjacent walls with m bridge segments, wherein the spacing between two adjacent repeating multi-bridge fibers is about x to (l−m−1)x (e.g., an integer multiple of x), wherein l is any integer greater than 3 (such as less than 1,000,000, such as about 10 to about 10,000, such as about 10 to about 5,000, such as about 10 to about 1,000, such as about 50, 100, 500, 1,000, or 5,000, etc.), and x is the spacing between two adjacent walls (see e.g., FIG. 16), and n is m+1. In some examples, one or more of the reinforcing layers (such as at least about 10%, 20%, 30%, 40%, 45%, 50%, 55%, 60%, 65%, 70%, 75%, 80%, 85%, 90%, 95%, 96%, 97%, 98%, or 99%, or all of the reinforcing layers) of the scaffold include repeating multi-bridge fibers.

In some aspects, the scaffold includes, for one or more of the multi-bridge fibers (such as at least about 10%, 20%, 30%, 40%, 45%, 50%, 55%, 60%, 65%, 70%, 75%, 80%, 85%, 90%, 95%, 96%, 97%, 98%, or 99%, or all of the multi-bridge fibers), a corresponding mirroring multi-bridge fiber, wherein a bridge segment of the multi-bridge fiber and a bridge segment of the corresponding mirroring multi-bridge fiber pass each other (with or without contact) (see, e.g., FIG. 16). In some examples, mirroring multi-bridge fibers are in a layer adjacent to the layer of their corresponding multi-bridge fibers. In some examples, mirroring multi-bridge fibers are in a layer that is not adjacent to the layer of their corresponding multi-bridge fibers. For instance, the mirroring multi-bridge fibers may be located at least one (such as at least 2, 3, 4, 5 . . . to up to the total number of layers minus the layer number of their corresponding fibers) layer apart from the layer of their corresponding multi-bridge fibers. In some examples, one or more of the reinforcing layers (such as at least about 10%, 20%, 30%, 40%, 45%, 50%, 55%, 60%, 65%, 70%, 75%, 80%, 85%, 90%, 95%, 96%, 97%, 98%, or 99%, or all of the reinforcing layers) of the scaffold include pairs of mirroring multi-bridge fibers.

In some aspects, the scaffold may include along its perimeter a reinforcing structure that provides improved mechanical stability and thus facilitates handling during fabrication or implantation.

In some aspects, the scaffold includes a plurality of substantially identical, three-dimensional structural units, such that successive fabrication runs reproduce the same three-dimensional pattern while extending the structure in one or more spatial directions to form the scaffold. Thus, the scaffold is not limited to a planar configuration, but may instead be tubular (or other shape) or conform to a complex, arbitrary three-dimensional shape.

In some aspects, the wall fibers and/or reinforcing fibers include a biocompatible material, such as a biocompatible polymer. In some examples, the wall fibers and/or reinforcing fibers include poly(α-hydroxy acids), poly(orthoesters), sugars, dextrans, PCL, PDO, PVA, PLGA, PLCL, PGA, PLA, PLLA, PEGDA, polyurethanes, nylon, thermoplastic elastomers, liquid crystal elastomers, PEEK, PEKK, or any combination thereof. In some examples, the wall fibers and reinforcing fibers include the same material, while in other examples, the wall fibers and reinforcing fibers include different materials.

In some aspects, the scaffold has an apparent Young's modulus of about 10 KPa to about 5 GPa under tensile loading, e.g., when analyzed as one homogeneous and continuous body. The Young's modulus will depend on the fiber material. In some examples, when the material includes PCL, the Young's modulus is about 5 MPa to about 15 MPa, such as about 7 MPa to about 15 MPa, along the direction of the walls. In some examples, when the material includes fibrin, the Young's modulus is about 1 KPa to about 15 KPa. In some examples, when the material includes poly-l-lactic acid (PLLA), the Young's modulus is about 1 GPa to about 15 GPa. In some examples, when the material includes polyetherether ketone (PEEK), the Young's modulus is about 3 GPa to about 4 GPa. In some examples, when the material includes poly(ethylene glycol) diacrylate (PEGDA), the Young's modulus is about 10 KPa to about 4 MPa. In some examples, when the material includes poly(dioxanone) (PDO), the Young's modulus is about 10 MPa to about 1 GPa.

In some aspects, the scaffold has a yield point stress of about 5 KPa to about 150 MPa. The yield point stress will depend on the fiber material. In some examples, when the material includes PCL, the yield point stress is about 450 KPa to about 800 KPa, such as about 500 KPa to about 750 KPa, along the direction of the walls. In some examples, when the material includes PLLA, the yield point stress is about 60 MPa to about 70 MPa. In some examples, when the material includes PEEK, the yield point stress is about 90 MPa to about 115 MPa. In some examples, when the material includes PDO, the yield point stress is about 1 MPa to about 40 MPa. In some examples, when the material includes PEGDA, the yield point stress is about 10 MPa to about 25 MPa.

In some aspects, the scaffold has a yield strain of about 0.05% to about 300%. The yield strain will depend on the fiber material. In some examples, when the material includes PCL, the yield strain is no more than 10%, or no more than 7.5%, such as about 5.0% to about 10%, such as about 5.0% to about 7.5%, along the direction of the parallel walls. In some examples, when the material includes PEEK, the yield strain is about 20% to about 45%. In some examples, when the material includes PDO, the yield strain is about 50% to about 150%. In some examples, when the material includes PLLA, the yield strain is about 1% to about 10%. In some examples, when the material includes PEGDA, the yield strain is about 30% to about 300%. Suitable methods for measuring these properties are known in the art.

### IV. Compositions

Also provided are compositions including i) the ultrafine-fiber or microfiber scaffold disclosed herein, and ii) a hydrogel, and/or cells.

In some aspects, the compositions include the scaffold and a hydrogel. In some embodiments, a scaffold may be composited with a hydrogel by incorporating or embedding the hydrogel material within or around the scaffold to create a composite structure that combines the mechanical stability of the scaffold with the hydration and biofunctional properties of the hydrogel. In some examples, the hydrogel precursor solution can be infused into the porous network of the scaffold and subsequently crosslinked in situ to form a continuous, hydrated phase throughout the scaffold. Alternatively, the hydrogel may be applied as a coating or interpenetrating network that integrates with the scaffold surface or internal architecture. In another embodiment, the scaffold and hydrogel may be composited through a simple layering approach, in which the hydrogel is placed directly on top of, or in contact with, the scaffold without substantial interpenetration or mixing of the materials. For example, a pre-formed or in situ-gelled hydrogel layer may be deposited onto the surface of the scaffold to create a stratified construct. The hydrogel layer can serve as a bioactive interface, moisture reservoir, or cell-encapsulating medium, while the underlying scaffold provides mechanical support and structural definition.

In some examples, the hydrogel may include any suitable (e.g., biocompatible) material, including i) natural polymers, such as collagen, gelatin, hyaluronic acid, chitosan, alginate, agarose, fibrin, silk fibroin, dextran, or carrageenan; ii) synthetic polymers, such as poly(ethylene glycol) (PEG), poly(vinyl alcohol) (PVA), polyacrylamide (PAAm), poly(N-isopropylacrylamide) (PNIPAAm), poly(2-hydroxyethyl methacrylate) (PHEMA), polyurethane (PU); iii) composite or hybrid materials, such as PEG-gelatin, PEG-hyaluronic acid, alginate-collagen, PEG-fibrin, nanocomposite hydrogels (e.g., incorporating nanoclay, silica nanoparticles, or graphene oxide), bioactive glass-polymer composites, or calcium phosphate-polymer composites; or iv) any combination thereof. In some examples, the hydrogel includes type I collagen, gelatin methacrylate, fibrin, agarose, laminin, hyaluronic acid, alginate Matrigel, polyethylene glycol, or any combination thereof.

In some examples, the hydrogel may further include living cells dispersed or encapsulated within its matrix to promote biological activity and/or tissue regeneration. The cells may be seeded into the hydrogel before, during, or after gelation, depending on the gelation mechanism and desired spatial distribution. Suitable cell types may include stem cells, progenitor cells, or differentiated cells relevant to the intended application, such as osteoblasts, chondrocytes, fibroblasts, or endothelial cells. In some examples, the cells are musculoskeletal cells, including tenocytes, ligamentocytes, osteoblasts, myocytes, osteocytes, osteoclasts, bone lining cells, chondroblasts, chondrocytes, progenitor cells, skeletal muscle fibers, satellite cells, myoblasts, myosatellite cells, fibroadipogenic progenitors, myofibroblasts, type A synoviocytes, type B synoviocytes, fibroblasts, mesenchymal stem/stromal cells, induced pluripotent stem cells, endothelial cells, pericytes, and macrophages. In some examples, the cells include skeletal muscle cells, ligament cells, tendon cells, cartilage cells, bone cells, vascular cells, fibrogenic cells, neural cells, cardiac cells, or any combination thereof. The hydrogel composition can be formulated to provide a biocompatible and supportive microenvironment that facilitates cell survival, proliferation, and differentiation. Incorporating cells within the hydrogel phase allows the composite construct to serve as a bioactive scaffold capable of stimulating tissue growth and integration with host tissues.

In some aspects, the compositions include the scaffold and cells. A scaffold and cells may be combined to form a construct that supports tissue regeneration without the use of a hydrogel component. The scaffold provides a structural framework that mimics the extracellular matrix, offering mechanical stability, defined porosity, and surface features conducive to cell attachment and proliferation. Cells can be seeded onto or within the scaffold, where they adhere, spread, and interact with the material surface. Over time, the cells may deposit their own extracellular matrix and remodel the scaffold as it degrades or integrates with the host tissue. Suitable cell types may include stem cells, progenitor cells, or differentiated cells relevant to the intended application, such as osteoblasts, myocytes, chondrocytes, fibroblasts, or endothelial cells. In some examples, the cells are musculoskeletal cells, including tenocytes, ligamentocytes, osteoblasts, osteocytes, osteoclasts, bone lining cells, chondroblasts, chondrocytes, progenitor cells, skeletal muscle fibers, satellite cells, myoblasts, myosatellite cells, fibroadipogenic progenitors, myofibroblasts, type A synoviocytes, type B synoviocytes, fibroblasts, mesenchymal stem/stromal cells, induced pluripotent stem cells, pericytes, and macrophages. In some examples, the cells include skeletal muscle cells, ligament cells, tendon cells, cartilage cells, bone cells, vascular cells, fibrogenic cells, neural cells, cardiac cells, or any combination thereof.

### IV. Methods of Manufacturing

Provided are novel methods of manufacturing ultrafine-fiber or microfiber scaffolds utilizing electrohydrodynamic printing (EHD) (such as melt electrowriting (MEW)), where an electric field is used to control the ejection and deposition of a material. By adjusting the printing path angles, the jumping of a fiber can be controlled to form reinforcing fibers with one or more bridge segments, which unexpectedly provide superior properties advantageous in cell and tissue culturing as demonstrated herein. The jumping between walls is induced by the mechanical pulling force of a nozzle when its path is offset at an angle relative to the existing walls. As the nozzle guides the fiber along the printing path, the electrostatic charge acts to maintain fiber deposition onto the existing walls, thereby inducing the formation of an anchor segment before the fiber crosses to an adjacent wall.

Prior MEW techniques have been limited by spontaneous fiber bridging, which occurs as an uncontrolled defect due to electrostatic attractions and residual charges, often restricting precise scaffold architectures and leading to unintended deviations between adjacent fibers. Unlike these spontaneous bridging defects in prior MEW, the present disclosure provides a controlled offset jetting method that intentionally induces multi-bridge reinforcements, enabling the formation of stable, multi-segment reinforcing fibers that connect multiple adjacent walls for enhanced structural integrity and alignment in tissue engineering scaffolds. It is disclosed herein, for the first time, that MEW can be utilized to control the formation of reinforcing fibers that bridge at least four adjacent walls with at least three bridge segments, providing advantageous properties. In contrast, prior art has regarded the jumping as an uncontrollable and undesirable defect that occurs between neighboring two or three walls.

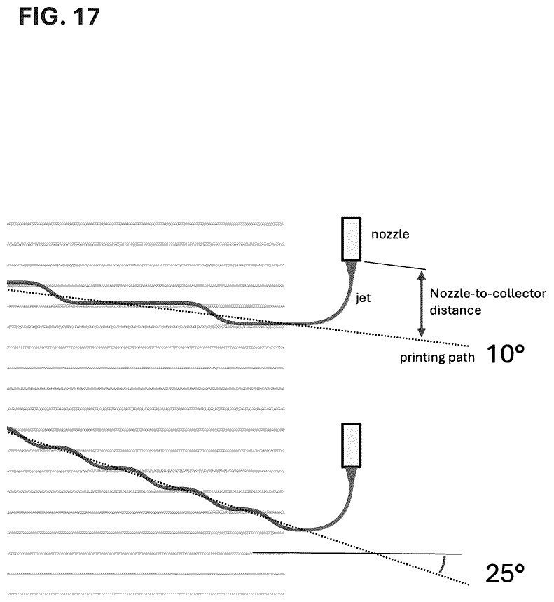

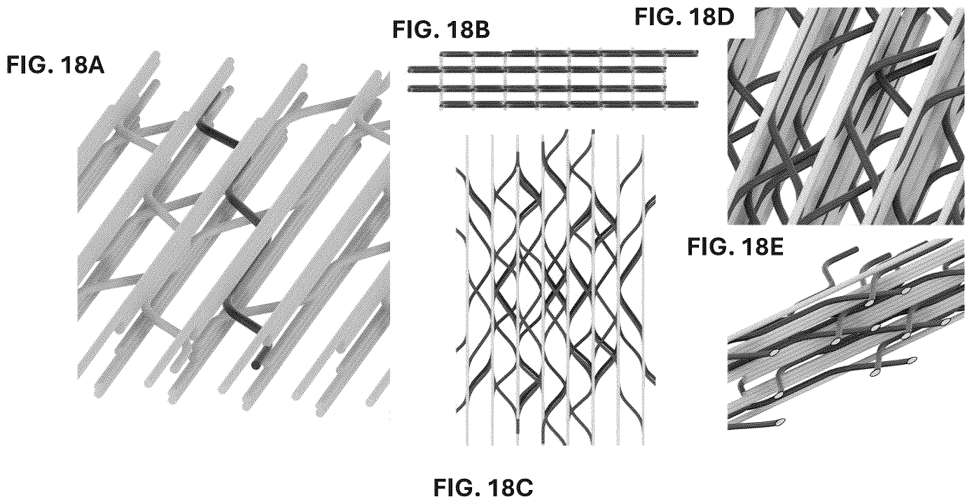

Provided are methods of producing an ultrafine-fiber or microfiber scaffold using EHD, including: (a) providing a first set of wall fibers (e.g., jetted by EHD) that are substantially parallel to each other, and (b) EHD jetting along a straight jetting path that is offset relative to the wall fiber direction (0° axis) at an angle of less than 90° (see, e.g., FIGS. 15A-15C and 17). The angle encompassing both angles formed on either side of the wall direction, which may be indicated with “±”. In some examples, the angle is greater than 3° or about 3°, such as about 3° to about 45°, about 3° to about 40°, about 3° to about 35°, about 3° to about 30°, about 3° to about 25°, about 3° to about 20°, about 3° to about 15°, about 3° to about 10°, about 3° to about 9°, about 3° to about 8°, about 3° to about 7°, about 3° to about 6°, or about 3° to about 5°.

In some aspects, step (a) includes providing a first set of wall fibers comprising q wall fibers jetted by EHD that are substantially parallel to each other, wherein adjacent wall fibers in the first set are spaced at x, and q is any integer greater than 3 (such as less than 1,000,000, such as less than 100,000, such as about 10 to about 10,000, such as about 10 to about 5,000, such as about 10 to about 1000, such as about 50 to about 150, or about 50, 100, 500, 1,000, or 5,000, etc.); and step (b) includes EHD jetting at least one reinforcing fiber along a first straight diagonal jetting path that begins at the same point as a first wall fiber of the first set, and ends at the same point as a second wall fiber of the first set, wherein the first wall fiber and second wall fiber are separated by p, wherein p=any value from 3x to (q−1)x. See., e.g., FIGS. 15A-15C. In some examples, x is about 40 μm to about 500 μm, such as about 40 μm to about 400 μm, about 40 μm to about 350 μm, about 40 μm to about 300 μm, about 40 μm to about 250 μm, about 40 μm to about 200 μm, about 40 μm to about 150 μm, about 50 μm to about 500 μm, about 50 μm to about 400 μm, about 50 μm to about 350 μm, about 50 μm to about 300 μm, about 50 μm to about 250 μm, about 50 μm to about 200 μm, about 50 μm to about 150 μm, about 60 μm to about 500 μm, about 60 μm to about 400 μm, about 60 μm to about 350 μm, about 60 μm to about 300 μm, about 60 μm to about 250 μm, about 60 μm to about 200 μm, about 60 μm to about 150 μm, about 65 μm to about 500 μm, about 65 μm to about 400 μm, about 65 μm to about 350 μm, about 65 μm to about 300 μm, about 65 μm to about 250 μm, about 65 μm to about 200 μm, about 65 μm to about 150 μm, about 70 μm to about 500 μm, about 70 μm to about 400 μm, about 70 μm to about 350 μm, about 70 μm to about 300 μm, about 70 μm to about 250 μm, about 70 μm to about 200 μm, about 70 μm to about 150 μm, about 75 μm to about 500 μm, about 75 μm to about 400 μm, about 75 μm to about 350 μm, about 75 μm to about 300 μm, about 75 μm to about 250 μm, about 75 μm to about 200 μm, about 75 μm to about 150 μm, about 80 μm to about 500 μm, about 80 μm to about 400 μm, about 80 μm to about 350 μm, about 80 μm to about 300 μm, about 80 μm to about 250 μm, about 80 μm to about 200 μm, about 80 μm to about 150 μm, about 85 μm to about 500 μm, about 85 μm to about 400 μm, about 85 μm to about 350 μm, about 85 μm to about 300 μm, about 85 μm to about 250 μm, about 85 μm to about 200 μm, about 85 μm to about 150 μm, or about 100 μm. In some examples, x between any two adjacent walls is the same.

In some aspects, the methods further include: repeating step (b) with a second straight diagonal jetting path that is parallel to the first straight diagonal jetting path, wherein the second and first straight diagonal jetting paths are separated by any value from x to (q−p−1)x.

In some aspects, the methods further include: (c) EHD jetting at least one reinforcing fiber along a first straight diagonal mirroring jetting path that mirrors the first straight diagonal jetting path.

In some aspects, the methods further include repeating step (c) with a second straight diagonal mirroring jetting path that is parallel to the first straight diagonal mirroring jetting path, wherein the second and first straight diagonal mirroring jetting paths are separated by any value from x to (q−p−1)x.

In some aspects, the methods further include, before step (c): EHD jetting a second set of wall fibers having a total of q wall fibers that are substantially parallel to each other and substantially aligned with the wall fibers in the first set, wherein adjacent wall fibers in the second set are spaced at x.

In some aspects, the methods include EHD jetting a first reinforcing layer including repeating reinforcing fibers, wherein the layer is jetted with a first set of diagonal straight jetting paths that are offset at the same angle relative to the wall fiber direction, wherein the angle is greater than 3° or about 3°, such as about 3° to about 45°, about 3° to about 40°, about 3° to about 35°, about 3° to about 30°, about 3° to about 25°, about 3° to about 20°, about 3° to about 15°, about 3° to about 10°, about 3° to about 9°, about 3° to about 8°, about 3° to about 7°, about 3° to about 6°, or about 3° to about 5°. In some examples, the methods further include EHD jetting a second reinforcing layer including repeating reinforcing fibers each mirroring one of the reinforcing fibers of the first reinforcing layer, wherein the second layer is jetted with a second set of diagonal straight jetting paths each mirroring one of the jetting paths of the first set. In some examples, the methods further include jetting one or more wall layers before jetting the first reinforcing layer, between jetting the first and second reinforcing layers, and/or after jetting the second reinforcing layer.

The different steps can be combined in any sequence and repeated in any number of times, to provide the scaffold structures disclosed herein.

In some aspects, the wall fibers constitute about 50.0% to about 99.9% by weight of the microfiber scaffold (such as about 50% to about 99%, about 55% to about 99%, about 60% to about 99%, about 65% to about 99% about 70% to about 99%, about 75% to about 99%, about 80% to about 99%, about 85% to about 99%, about 90% to about 99%, about 50% to about 95%, about 55% to about 95%, about 60% to about 95%, about 65% to about 95% about 70% to about 95%, about 75% to about 95%, about 80% to about 95%, or about 85% to about 95%), and/or the reinforcing fibers constitute about 0.1% to about 50.0% by weight of the microfiber scaffold (such as about 1% to about 50%, about 1% to about 45%, about 1% to about 40%, about 1% to about 35% about 1% to about 30%, about 1% to about 25%, about 1% to about 20%, about 1% to about 15%, about 1% to about 10%, about 5% to about 50%, about 5% to about 45%, about 5% to about 40%, about 5% to about 35% about 5% to about 30%, about 5% to about 25%, about 5% to about 20%, or about 5% to about 15%). In some examples, the scaffold includes only wall fibers and reinforcing fibers.

In some aspects, the ratio of the number of reinforcing fibers to the number of the wall fibers is about 1:1,000,000 to about 1:1 (reinforcing fibers:wall fibers) (such as about 1:100,000 to about 1:10, about 1:10,000 to about 1:10, about 1:1,000 to about 1:10, about 1:500 to about 1:10, about 1:400 to about 1:10, about 1:300 to about 1:10, about 1:250 to about 1:10, about 1:200 to about 1:10, about 1:150 to about 1:10, about 1:100 to about 1:10, about 1:90 to about 1:10, about 1:80 to about 1:10, about 1:70 to about 1:10, about 1:60 to about 1:10, about 1:50 to about 1:10, about 1:40 to about 1:10, about 1:30 to about 1:10, about 1:20 to about 1:10, about 1:50 to about 1:5, about 1:40 to about 1:5, about 1:30 to about 1:5, about 1:20 to about 1:5, about 1:15 to about 1:5, or about 1:5 to 1:1).

In some aspects, the ratio of the number of reinforcing layers to the number of wall layers is about 1:1,000 to about 1:1 (reinforcing layers:wall layers), such as about 1:1,000 to about 1:3, about 1:1,000 to about 1:5, about 1:1,000 to about 1:8, about 1:1,000 to about 1:10, about 1:500 to about 1:3, about 1:500 to about 1:5, about 1:500 to about 1:8, about 1:500 to about 1:10, about 1:400 to about 1:3, about 1:400 to about 1:5, about 1:400 to about 1:8, about 1:400 to about 1:10, about 1:300 to about 1:3, about 1:300 to about 1:5, about 1:300 to about 1:8, about 1:300 to about 1:10, about 1:200 to about 1:3, about 1:200 to about 1:5, about 1:200 to about 1:8, about 1:200 to about 1:10, about 1:100 to about 1:3, about 1:100 to about 1:5, about 1:100 to about 1:8, about 1:100 to about 1:10, about 1:50 to about 1:3, about 1:50 to about 1:5, about 1:50 to about 1:8, about 1:50 to about 1:10, about 1:30 to about 1:3, about 1:30 to about 1:5, about 1:30 to about 1:8, about 1:30 to about 1:10, about 1:5 to about 1:1, about 2:9 to about 1:1, about 1:4 to about 1:1, about 2:7 to about 1:1, about 1:3 to about 1:1, about 2:5 to about 1:1, or about 2:5. In some examples, the reinforcing layers are regularly spaced in the scaffold. For instance, a reinforcing layer is introduced for every 1 to up to ½ of the total number of wall layers (such as 1, 2, 3, 4, 5 . . . 100 . . . 1000 . . . ) wall layers. In some examples, the scaffold includes only reinforcing layers and wall layers.

In some aspects, each of the wall fibers and reinforcing fibers have a diameter of less than about 100 μm (such as less than about 90 μm, about 80 μm, about 70 μm, about 65 μm, about 60 μm, about 55 μm, about 50 μm, about 45 μm, about 40 μm, about 35 μm, about 30 μm, about 25 μm, about 20 μm, about 15 μm, about 14 μm, about 13 μm, about 12 μm, or about 11 μm), or more than about 0.1 μm (such as more than about 0.2 μm, about 0.3 μm, about 0.4 μm, about 0.5 μm, about 0.6 μm, about 0.7 μm, about 0.8 μm about 0.9 μm, about 1 μm, about 1.5 μm, about 2 μm, about 2.5 μm, about 3 μm, about 3.5 μm, about 4μm, about 4.5 μm, or about 5 μm), or both (such as about 0.1 μm to about 100 μm, about 0.1 μm to about 50 μm, about 0.1 μm to about 40 μm, about 0.1 μm to about 30 μm, about 0.1 μm to about 20 μm, about 0.1 μm to about 15 μm, about 0.1 μm to about 12 μm, about 0.1 μm to about 10 μm, about 0.5 μm to about 100 μm, about 0.5 μm to about 50 μm, about 0.5 μm to about 40 μm, about 0.5 μm to about 30 μm, about 0.5 μm to about 20 μm, about 0.5 μm to about 15 μm, about 0.5 μm to about 12 μm, about 0.5 μm to about 10 μm, about 1 μm to about 100 μm, about 1 μm to about 50 μm, about 1 μm to about 40 μm, about 1 μm to about 30 μm, about 1 μm to about 20 μm, about 1 μm to about 15 μm, about 1 μm to about 12 μm, or about 1 μm to about 10 μm). In some examples, the diameter of each of the wall fibers and reinforcing fibers is about the same.

In some aspects, the wall fibers and/or reinforcing fibers include a biocompatible material, such as a biocompatible polymer. In some examples, the wall fibers and/or reinforcing fibers include poly(α-hydroxy acids), poly(orthoesters), sugars, dextrans, PCL, PDO, PVA, PLGA, PLCL, PGA, PLA, polyurethanes, nylon, thermoplastic elastomers, liquid crystal elastomers, PEEK, PEKK, or any combination thereof.

In some aspects, the EHD is MEW.

In MEW, the polymer is typically supplied in solid filament or powder form and melted within a temperature-controlled nozzle or syringe barrel. The processing temperature is selected above the polymer's melting or softening point to achieve a stable, continuous jet without thermal degradation. This temperature depends on the specific material used; for example, poly(ε-caprolactone) (PCL) is commonly processed at approximately 65-90° C., while higher-melting polymers such as poly(L-lactic acid) (PLLA) or poly(ether-ether-ketone) (PEEK) may require 150-350° C. In general, the preferred temperature range for MEW operation is one that provides a viscosity low enough for controlled jet formation yet high enough to maintain structural fidelity upon deposition. The precise temperature window is therefore material-dependent and determined experimentally to balance flow stability, jet control, and fiber solidification behavior.

In MEW, the applied voltage potential can influence the electric field strength between the nozzle and the collector, thereby affecting jet formation, stability, and deposition behavior, including the bridging of fibers across adjacent walls. The voltage can be tuned to generate a stable, focused jet that can span the gap between fiber walls without premature solidification, collapse, or instability (e.g., whipping or beading). In the examples described herein, an applied voltage potential of about 5.5 kV was used to achieve reliable bridging with PCL, but suitable voltages for bridge formation can range from about 2 kV to about 10 kV, such as from about 4 kV to about 7 kV, depending on the polymer viscosity, nozzle-to-collector distance, and other factors. Voltages in this range promote electrostatic autofocusing, facilitating bridge formation.

Several other parameters can be adjusted to optimize bridging behavior, including the nozzle-to-substrate gap (typically about 2 mm to about 10 mm, e.g., about 4 mm as used in one of the examples, to balance jet stability and fiber positioning precision), fiber diameter (generally about 1 μm to about 100 μm, with smaller diameters enhancing bridging behavior due to a more flexible fiber), polymer flow rate from the nozzle (controlled via applied pressure, e.g., about 0.05 bar to about 5 bar, such as about 0.15 bar to about 0.5 bar, to ensure consistent jet volume without overflow or starvation), nozzle gauge (about 20 gauge to about 30 gauge, such as about 22 gauge to about 26 gauge), and/or the ratio of the collector translation speed to the jet speed (typically about 1:1 to about 5:1, such as about 1:1.5 to 1:2, where ratios below 1:1 are prohibited to prevent uncontrolled fiber coiling or buckling). Environmental conditions, such as temperature (e.g., about 20° C.) and humidity, may also be tuned. Optimal bridging may be achieved by iteratively adjusting these parameters to maintain a stable Taylor cone and jet trajectory.

Also provided are ultrafine-fiber or microfiber scaffolds produced by the method disclosed herein.

### IV. Methods of Cell Culture, Tissue Engineering, and Treatment

Provided are methods of culturing cells, including culturing cells in the presence of a ultrafine-fiber or microfiber scaffold disclosed herein in a culture medium under conditions sufficient for the cells to grow. The culture medium may be any suitable medium capable of supporting the viability and proliferation of the selected cell type, such as a basal medium (e.g., Dulbecco's Modified Eagle Medium (DMEM), Minimum Essential Medium (MEM), or RPMI 1640) optionally supplemented with serum, growth factors, hormones, amino acids, antibiotics, or other additives known in the art. The culture conditions may include maintaining the cells at a temperature of approximately 35-38° C. in a humidified atmosphere containing about 5% CO₂, although other temperature or gas conditions may be used depending on the specific cell type. The medium may be static or dynamically perfused through or across the scaffold to promote nutrient exchange and waste removal. As demonstrated herein, the scaffold provides structural and topographical cues that facilitate cell attachment, alignment, differentiation, or tissue formation under suitable culture conditions.

Suitable cell types may include stem cells, progenitor cells, or differentiated cells relevant to the intended application, such as osteoblasts, myocytes, chondrocytes, fibroblasts, or endothelial cells. In some examples, the cells are musculoskeletal cells, including tenocytes, ligamentocytes, osteoblasts, osteocytes, osteoclasts, bone lining cells, chondroblasts, chondrocytes, progenitor cells, skeletal muscle fibers, satellite cells, myoblasts, myosatellite cells, fibroadipogenic progenitors, myofibroblasts, type A synoviocytes, type B synoviocytes, fibroblasts, mesenchymal stem/stromal cells, and induced pluripotent stem cells. In some examples, the cells include skeletal muscle cells, ligament cells, tendon cells, cartilage cells, bone cells, vascular cells, fibrogenic cells, neural cells, cardiac cells, or any combination thereof.

In some aspects, the methods further include the use of a hydrogel. Suitable materials to form a hydrogel include any biocompatible material, including i) natural polymers, such as collagen, gelatin, hyaluronic acid, chitosan, alginate, agarose, fibrin, silk fibroin, dextran, or carrageenan; ii) synthetic polymers, such as poly(ethylene glycol) (PEG), poly(vinyl alcohol) (PVA), polyacrylamide (PAAm), poly(N-isopropylacrylamide) (PNIPAAm), poly(2-hydroxyethyl methacrylate) (PHEMA), polyurethane (PU); iii) composite or hybrid materials, such as PEG-gelatin, PEG-hyaluronic acid, alginate-collagen, PEG-fibrin, nanocomposite hydrogels (e.g., incorporating nanoclay, silica nanoparticles, or graphene oxide), bioactive glass- polymer composites, or calcium phosphate-polymer composites; or iv) any combination thereof. In some examples, the hydrogel includes type I collagen, gelatin methacrylate, fibrin, agarose, laminin, hyaluronic acid, alginate Matrigel, polyethylene glycol, or any combination thereof.

In some embodiments, the methods include: i) contacting the ultrafine-fiber or microfiber scaffold with the cells and a hydrogel-forming material, thereby forming a three-dimensional composite structure wherein a hydrogel formed by the material encloses the cells and at least a part of the scaffold, and ii) culturing the three-dimensional composite structure in a culture medium under conditions sufficient for the cells to grow. For example, a hydrogel precursor solution containing one or more hydrogel-forming polymers and, optionally, crosslinking agents, photoinitiators, or ionic components is first prepared under sterile conditions. The precursor solution is mixed or gently combined with a suspension of cells to form a cell-laden prepolymer mixture. This mixture is then applied to or around the ultrafine-fiber or microfiber scaffold, allowing the fluid to infiltrate the fiber network or fill the spaces between fibers. The mixture is subsequently subjected to a polymerization or gelation step.

In some other embodiments, the ultrafine-fiber or microfiber scaffold is first coated or infiltrated with a hydrogel-forming material prior to the introduction of cells. The hydrogel-forming material may be applied to the scaffold in liquid or prepolymer form, allowing it to penetrate the fiber network or form a uniform coating over the scaffold surface. The material is then polymerized, crosslinked, or gelled under suitable conditions (for example, by exposure to light, temperature change, pH adjustment, or addition of a crosslinking agent) to form a stable hydrogel layer that is physically or chemically associated with the scaffold. After the hydrogel has solidified or reached the desired degree of gelation, cells are subsequently seeded onto or into the hydrogel-coated scaffold.

Provided are methods of culturing musculoskeletal cells, including: culturing cells in the presence of an ultrafine-fiber or microfiber scaffold in a culture medium under conditions sufficient for the cells to grow, wherein: the ultrafine-fiber or microfiber scaffold includes a plurality of wall fibers and a plurality of reinforcing fibers, wherein each reinforcing fiber includes at least two (such as at least four) anchor segments and at least one (such as at least three) bridge segments, wherein: the wall fibers and the anchor segments are substantially aligned forming a plurality of substantially parallel walls, and the bridge segments each extends across the space between two adjacent walls from one anchor segment to another of the same reinforcing fiber, forming an angle of less than 90° with the walls. In some examples, the musculoskeletal cells comprise skeletal muscle cells, ligament cells, tendon cells, cartilage cells, bone cells, vascular cells, and/or neural cells.

Provided are methods of treating musculoskeletal tissue injury in a subject, including: implanting an ultrafine-fiber or microfiber scaffold, or a composition including the ultrafine-fiber or microfiber scaffold and one or both of a hydrogel and musculoskeletal cells, at a site of musculoskeletal tissue injury in the subject, wherein: the ultrafine-fiber or microfiber scaffold includes a plurality of wall fibers and a plurality of reinforcing fibers, wherein each reinforcing fiber includes at least two (such as at least four) anchor segments and at least one (such as at least three) bridge segments, wherein: the wall fibers and the anchor segments are substantially aligned forming a plurality of substantially parallel walls, and the bridge segments each extends across the space between two adjacent walls from one anchor segment to another of the same reinforcing fiber, forming an angle of less than 90° with the walls. In some examples, the musculoskeletal cells include skeletal muscle cells, ligament cells, tendon cells, cartilage cells, bone cells, vascular cells, and/or neural cells.

Also provided are methods of treating tissue injury in a subject, including implanting a ultrafine-fiber or microfiber scaffold disclosed herein, or a composition including the scaffold disclosed herein at a site of tissue injury in the subject.

The scaffold may be implanted alone or in combination with a hydrogel, bioactive agent, or cell population to promote tissue repair, regeneration, or integration with the host tissue. In some embodiments, the scaffold serves as a temporary structural matrix that supports cellular infiltration, neovascularization, and extracellular matrix deposition. In other embodiments, the scaffold may be pre-seeded with autologous, allogeneic, or xenogeneic cells, including stem cells, progenitor cells, skeletal muscle cells, ligament cells, tendon cells, cartilage cells, bone cells, vascular cells, neural cells, or other cell types relevant to the injured tissue, prior to implantation. The scaffold and any associated materials may be formulated to degrade or resorb over time as new tissue forms. The implantation may be performed by direct placement, injection, or surgical insertion, depending on the size and location of the injury. In certain embodiments, the composition further includes growth factors, cytokines, or other biologically active molecules to enhance cell recruitment and tissue regeneration. The methods may be applied to repair or regenerate a variety of tissues, including but not limited to skin, muscle, nerve, bone, cartilage, tendon, vascular, or soft connective tissue.

### EXAMPLES

The following example is provided to illustrate particular features of certain aspects of the disclosure, but the scope of this disclosure should not be limited to those features exemplified.

### Overview

Novel microfiber scaffold and composite microfiber-hydrogel material were engineered using melt-electrowriting (MEW). The scaffolds were fabricated with high precision using poly(ε-caprolactone) (PCL), composited with collagen hydrogel, and then seeded with myoblasts. Aligned X scaffolds with cross-bridge reinforcements exhibited enhanced mechanical strength and continuous alignment without structural interruption that led to highly aligned and multinucleated cellular organization. The incorporation of collagen hydrogel in composite constructs improved cell seeding efficiency, viability, and metabolic activity compared to scaffolds alone. All scaffold designs provided fiber reinforcement that prevented hydrogel contraction over extended culture periods. Critically, the Aligned X composite constructs significantly increased myoblast differentiation and myotube maturation, evidenced by increased myosin heavy chain expression and myotube diameter. Overall, this composite microfiber-hydrogel approach provides a scalable, structurally stable, and highly aligned platform tailored for enhanced muscle tissue engineering applications, representing an advancement towards addressing clinical challenges associated with muscle injuries.

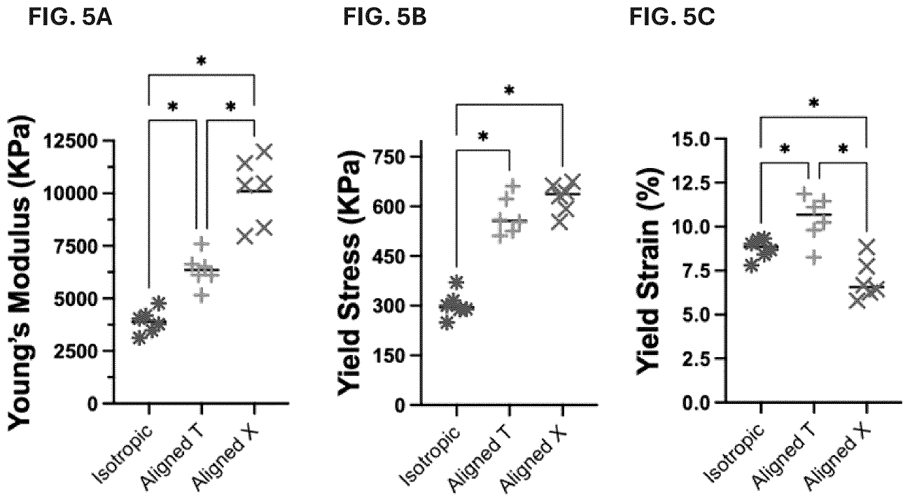

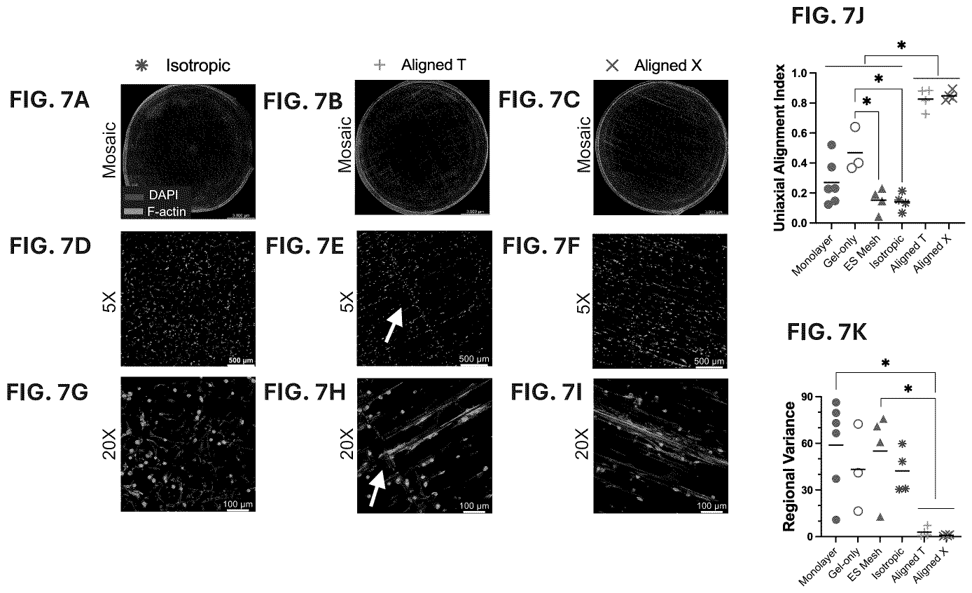

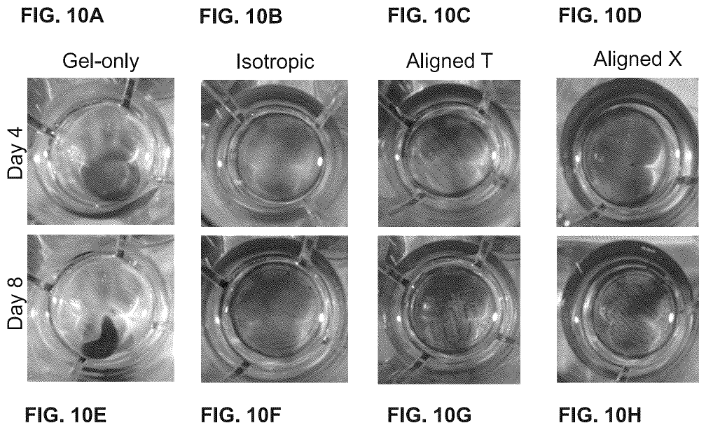

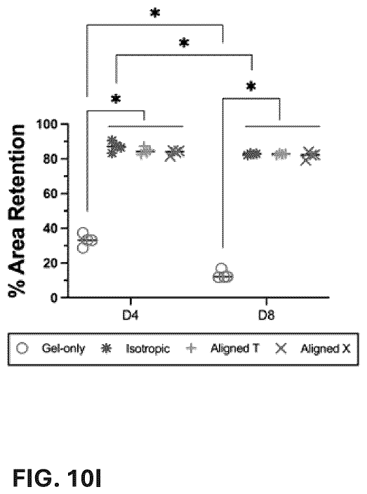

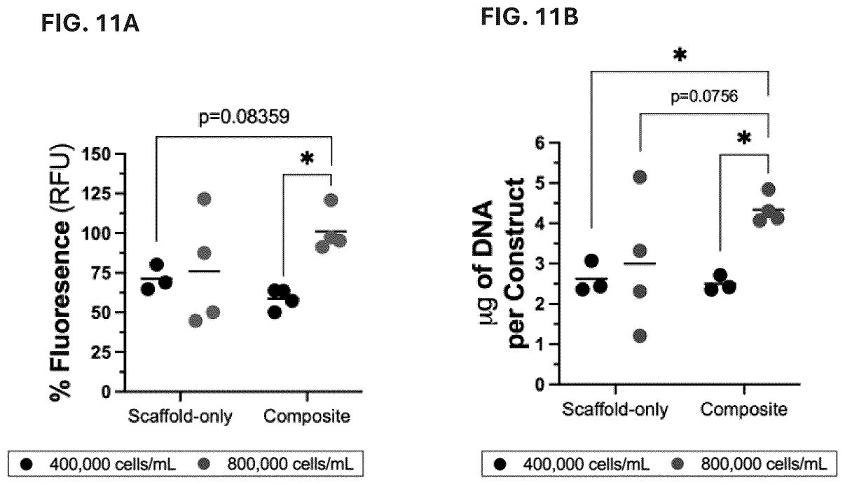

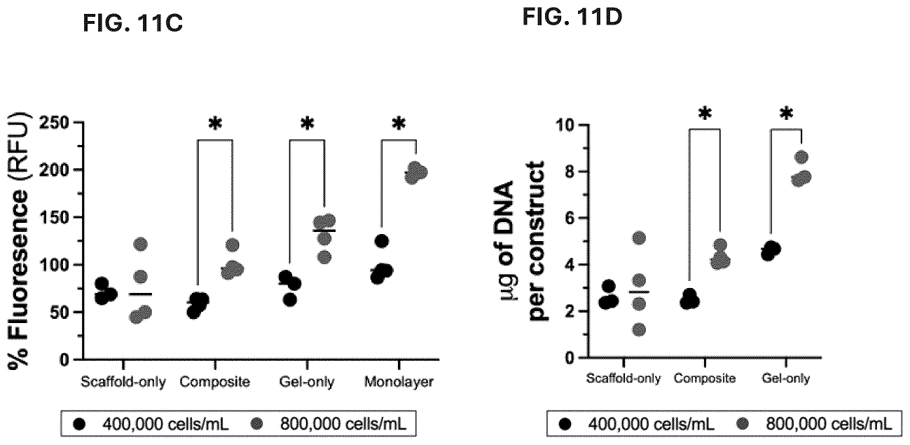

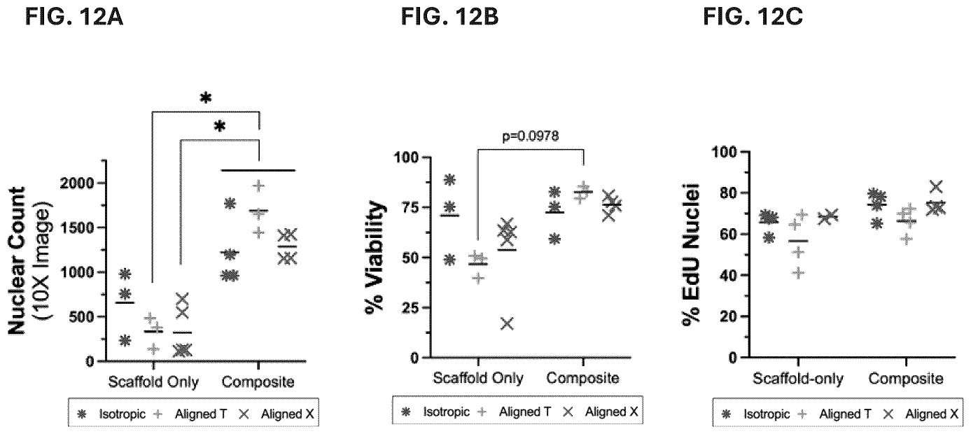

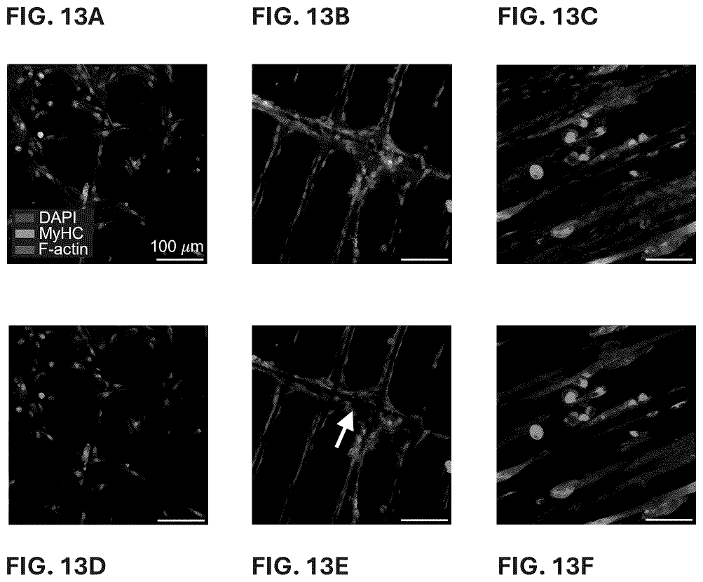

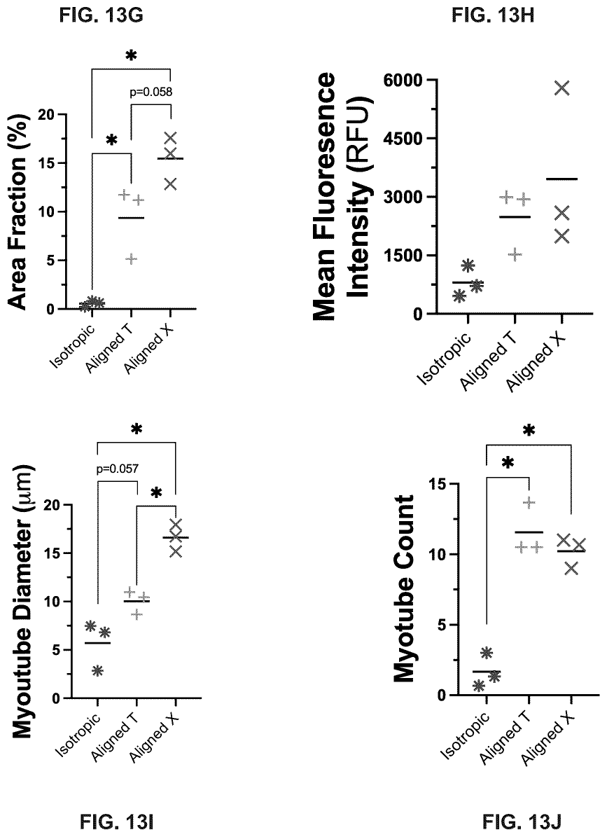

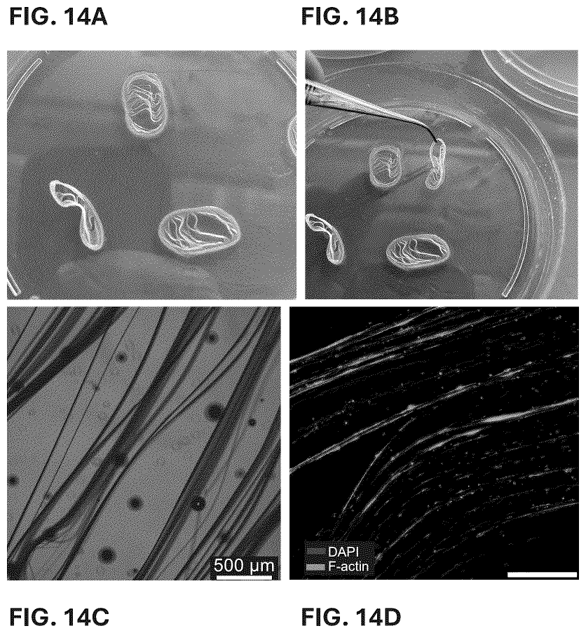

The designed microfiber architecture effectively regulated the cellular organization from the microscale (˜10 μm) to the macro scale (˜1 cm), modulated mechanical properties (FIGS. 5A-5C), and provided bulk structural stability (FIGS. 10A-10I). The hydrogel component improved cellular encapsulation efficiency, increasing cell retention, metabolic activity, and viability (FIGS. 11A-11D and 12A-12C). Composite constructs also demonstrate design versatility through three distinct scaffold architectures that change cellular organization and significantly influence myogenic cell development (FIGS. 7A-7K and 13A-13J).

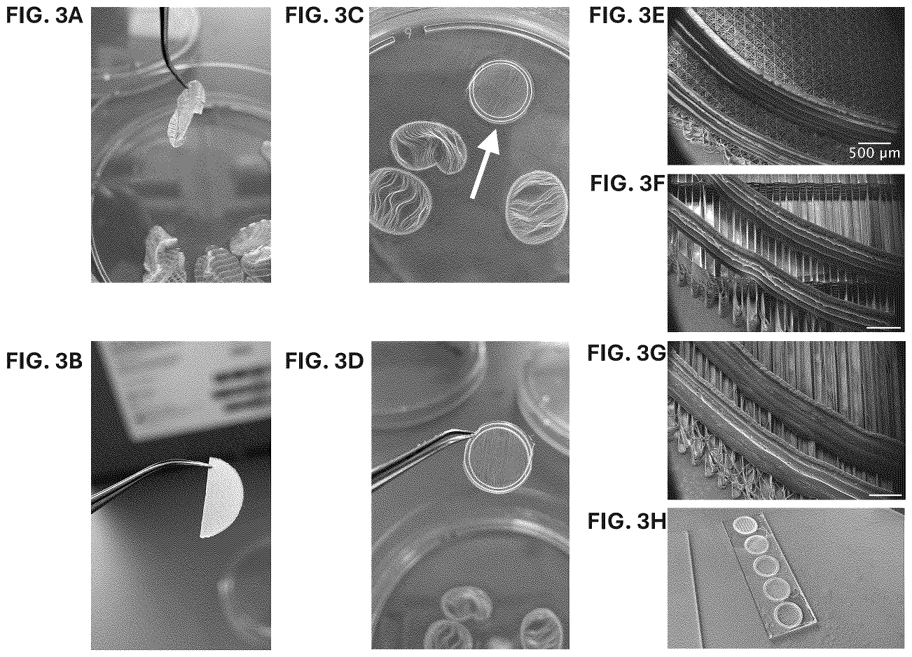

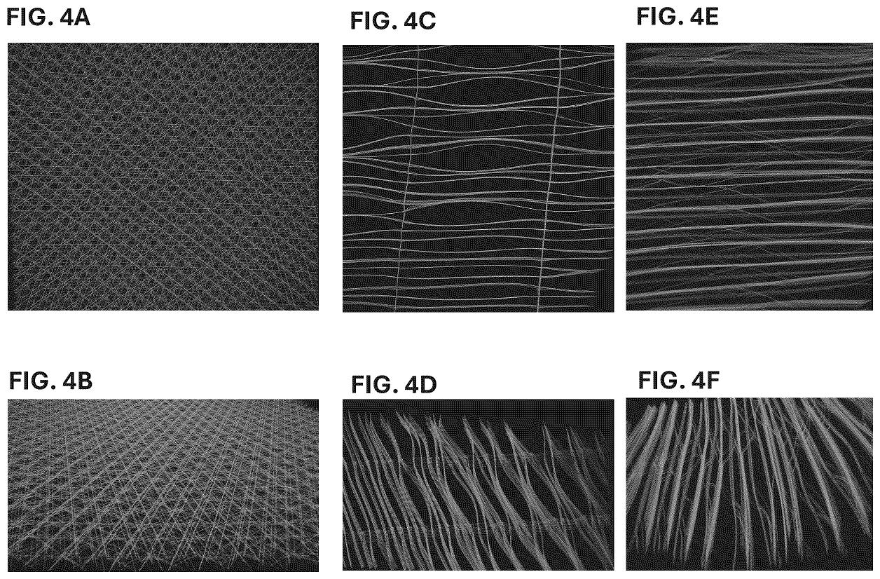

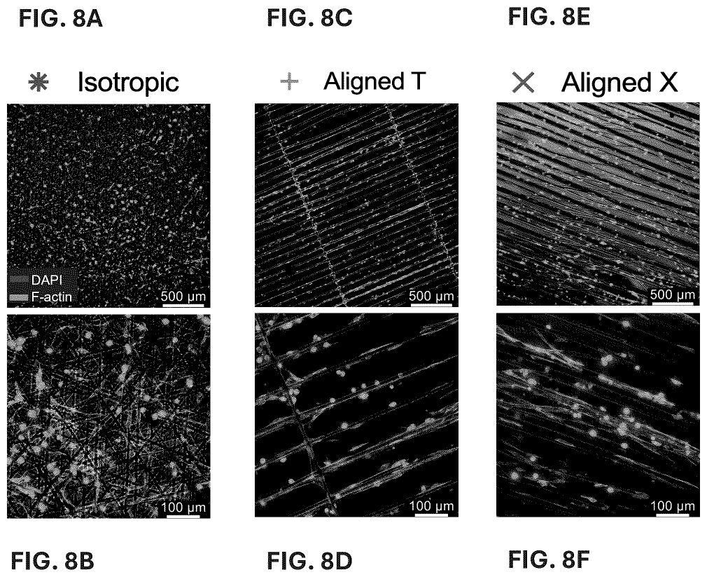

Among these, the Aligned X scaffold architecture enabled constructs with improved myoblast differentiation and myotube maturation. Large scaffolds including long fibrillar structures need structural reinforcements to maintain handling stability. Lack of reinforcements resulted in increased movement that lead to less cellular alignment (FIGS. 3C and 13A-13J). Perpendicular reinforcements have been previously utilized to address this problem. Yet, the reinforcements have deleterious effects on cellular organization (FIGS. 7E, 7H, 8C, and 8D), as they prevent long continuous myotube formation (FIG. 13E). Perpendicular reinforcements were also found to influence hydrogel contraction that led to phase separation (FIG. 10G). In contrast, the Aligned X architecture permitted continuous alignment over large distances with minimal effect on cellular organization as cells conformed to the scaffold geometry (FIGS. 7A-7K and 8A-8F). Further, the Aligned X architectures significantly increased expression of MyHC and myotube diameters (FIGS. 13A-13J). This is partly due to the increased cellular continuity without barriers to long myotube formation. The increased myotube maturation could also be due to increased microenvironmental structural integrity, as the Aligned X microfiber scaffolds maintained their geometry more than Aligned T scaffolds (FIGS. 4A-4F).

Scaffold mechanical properties were markedly affected by architectural changes alone, resulting in more than two-fold increase in yield stress from 3.89±0.575 MPa in the Isotropic group to 10.1±1.62 MPa in the Aligned X group (FIG. 5B), without changing scaffold mass. The magnitude of these moduli is similar to ex vivo tensile modulus of human levator ani muscles reported to be k=4.69±2.91 MPa, a linear scaling parameter of a power law constitutive model. Meanwhile, longitudinal extension of the extensor digitorum longus of rabbits, a hindlimb muscle, had a linear modulus of 447±97.7 Kpa. The mechanical properties of scaffolds can further be tailored by altering the microfiber diameter, the number of fibers, their spacing, or the polymer type. These scaffold variables therefore provide a large design space through which the mechanical properties can be adjusted to recapitulate specific tissue needs.

The current approach yielded constructs with cells populating most of the area along the microfiber walls by eight days of culture (FIGS. 7A-7K) and resulted in robust cellular differentiation by day 16 (FIGS. 13A-13J).

The utilization of microfiber scaffolds as a fiber reinforcement to soft matrices advances the design space and capabilities for engineered tissue constructs with micron-scale precision. Microfiber scaffolds disclosed herein dramatically altered cellular organization (FIGS. 7A-7K and 8A-8F), while stabilizing the bulk tissue geometry (FIGS. 10A-10I). MEW technology enables fabrication of micron-scale reinforcements over constructs larger than a centimeter. This enables design of muscle fiber geometries with multipennate geometry, or curvature to recapitulate muscles that traverse a joint. Alternatively, it may be used to introduce spatial heterogeneities by manufacturing scaffolds with subregions that are not supported with microfibers to model structural deficits in muscle tissue or introduce a higher concentration of cross-bridge reinforcements in one location to model the musculotendon junction.

### Example 1

### Materials and Methods

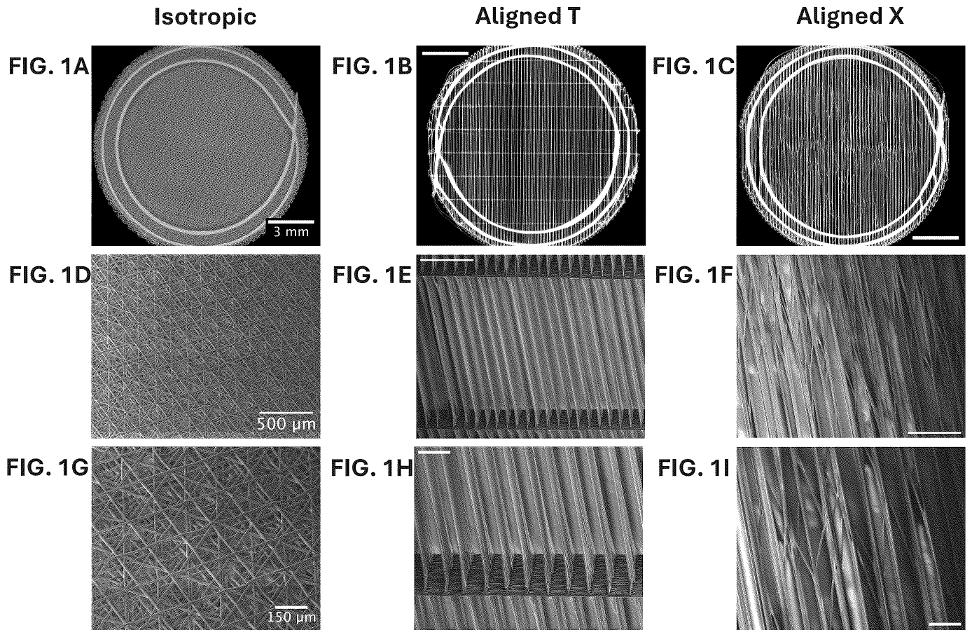

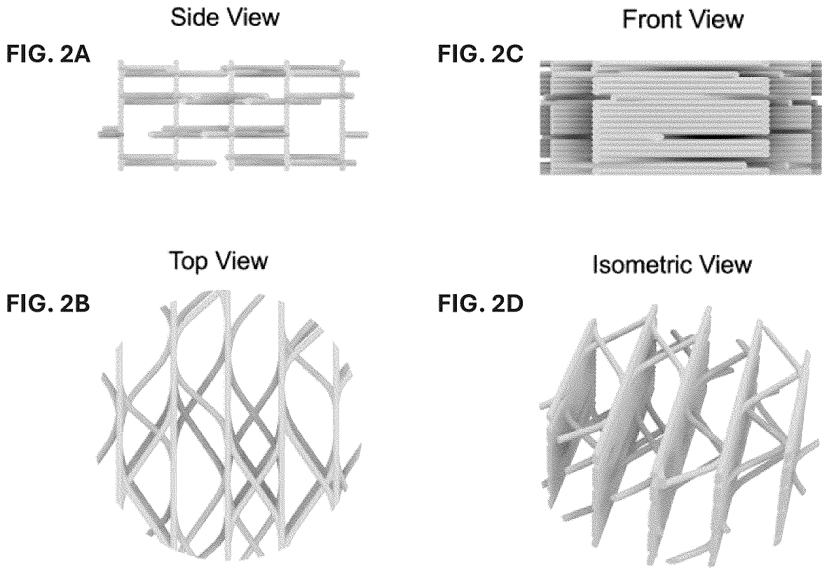

Microfiber Scaffolds: MEW scaffolds made from 10 μm microfibers were 3D-printed using poly(ε-caprolactone) (PCL) (PURAC PC12, Corbion Inc.). Fibers were deposited into a total of 20 layers with three different designs while keeping the total amount of extruded material consistent. The three scaffold architectures include “Isotropic” 5-fiber morphology (FIGS. 1A, 1D, and 1G), “Aligned T” with perpendicular running reinforcements (FIGS. 1B, 1E, and 1H), and “Aligned X” with cross-bridge reinforcements at an angle between 30-60° and semi-randomly dispersed throughout the scaffold plane (FIGS. 1C, 1F, and 1I). The scaffolds were fabricated in a circular shape with an overall diameter of 13.0 mm. The isotropic scaffold was previously used for an organoid model of skin, and contains spacing between each parallel track of fibers of 150 μm, with fibers running at 0°, 36°, 72°, 108°, and 144° (FIGS. 1A, 1D, and 1G). The Aligned T scaffold architecture was previously used for engineered articular cartilage, and employed 100 μm between aligned fiber walls and 1.5 mm between reinforcing perpendicular walls (FIGS. 1B, 1E, and 1H). The Aligned X architecture had 100 μm spacing between aligned fiber walls while cross-bridge reinforcements were accomplished by offsetting fiber printing directions by ±3° from the principal alignment axis in 8 of the 20 layers comprising each scaffold. The offset caused deposition of a fiber on previously laid material and led to “fiber bridging” to the adjacent fiber wall as the planar distance of the extruded jet and the original fiber wall increased. The number of layers was chosen by testing 3 different configurations with 5, 8 or 10 offset layers. The Aligned X scaffold with 8 bridging fiber layers interlaced uniformly with the aligned layers and qualitatively yielded the best combination of maintaining shape and porosity (FIGS. 1C, 1F, 1I, and FIGS. 2A-2D). All scaffolds included two reinforcement rings each from 4 layers of large (70-μm diameter) fibers along their perimeters which improved construct handling under wet and dry conditions by resisting folding, bending, and kinking (FIGS. 3A-3H). Scanning Electron Microscopy (SEM) was used to visualize and qualitatively compare the three scaffold designs. Scaffolds were sputter-coated with 7 nm of titanium prior to imaging with an SEM Everhart-Thornley detector (ThermoFisher Apreo 2, USA).

Scaffolds were fabricated using a custom-built MEW printer using the following processing and instrument parameters: a melt temperature of 75° C. was used to extrude to an electrically grounded 22 G nozzle (with 6.3 mm length) protruding 1 mm from the printhead. Nozzle-to-collector distance was fixed to 4 mm, and scaffolds were printed onto 1 mm thick glass microscopy slides resting on a metal collector with an applied voltage potential of 5.5 kV. Fibers with 10.0 μm were achieved by applying 0.17 bar pressure and 500 mm/min collector translation speed. Reinforcement rings along scaffold perimeter with 70.0 μm fiber diameter were printed by applying 3.0 bar pressure and 65 mm/min collector translation speed. Microfiber scaffolds with reinforcement rings were released from the glass slides by wetting with ethanol solution.

Mechanical Testing: Tensile properties of dry, acellular microfiber scaffolds were evaluated using a TA Electroforce 3220 mechanical tester equipped with a 1000 g load cell. Rectangular samples were carefully cut from circular scaffolds using surgical scissors to a target width of 4.0 mm. Actual measured widths of the scaffolds were not significantly different across the groups, averaging 4.63±0.55 mm, 4.25±0.94 mm, and 4.30±0.51 mm for the Isotropic, Aligned T, and Aligned X groups respectively. Scaffold lengths were measured between clamps upon mounting and were not significantly different with mean lengths of 9.47±0.90 mm, 8.25±0.99 mm, and 8.50±0.84 mm. Tests were performed to failure with a strain rate of 0.2 mm/s with the recorded signal of force data recorded at a minimum frequency of 10 Hz. Post-testing analysis utilized custom Matlab (MathWorks) code to calculate Young's modulus, yield stress, and yield strain. Nominal stress was computed by dividing the measured force by the sample's cross-sectional area, assuming uniform thickness due to consistent fiber layering and total scaffold mass. Strain was computed relative to initial sample length. Stress-strain curves were smoothed with a three-position moving window filter. Elastic modulus and yield parameters were numerically identified using the derivative of the stress-strain curves: the elastic region was defined as a drop of 20% of the peak slope, while yield points corresponded to the location slope decreased to 20% of the peak slope following linear elasticity. Young's modulus was determined by linear regression within the elastic region, and the yield stress/strain were recorded at the yield point.

Seeding Muscle Constructs with Microfiber Scaffolds: Composite muscle constructs were fabricated by embedding microfiber scaffolds within collagen hydrogels containing suspended cells. Microfiber scaffolds were sterilized in 70% ethanol for a minimum of 24 h, rinsed three times with 1× phosphate-buffered saline (PBS), and incubated in growth medium for a minimum of 12 hours at 37° C. C2C12 myoblasts cells (ATCC, cat. CRL1772) were suspended in type I collagen solution (Advanced Biomatrix; 1.5 mg/mL), at a density of 400,000 cells/mL, unless otherwise specified. 100 μL of collagen-cell suspension was pipetted onto scaffolds placed within ultra-low attachment 24 well plates (Costar, Fischer Scientific) and polymerized at 37° C.

Scaffold-only constructs, used as controls, were identically prepared and seeded with an equivalent total cell number (40,000 cells/scaffold). C2C12 myoblasts were directly pipetted onto microfiber scaffolds rather than within a collagen gel. For metabolic activity and DNA quantification assays, a higher seeding density of 800,000 cells/mL was additionally tested, with corresponding scaffold-only constructs seeded at 80,000 cells/scaffold for this condition.

Cell Culturing Conditions: Constructs were cultured initially in growth medium comprised of Dulbecco's Modified Eagle Medium (DMEM), 10% Fetal Bovine Serum (FBS), and 1% penicillin and streptomycin for 5 days. The medium was switched to differentiation medium (DMEM, 2% horse serum, and 1% penicillin/streptomycin) for 3 additional days, with media changes on odd days. For differentiation and maturation assays, the total culture duration was extended to 16 days.

Quantification of Cellular Alignment: Cellular alignment in composite microfiber-hydrogel constructs (Isotropic, Aligned T, and Aligned X) was evaluated after 8 days of culture. Additional control groups included monolayer culture on tissue culture plastic (5,000 cells/cm²), hydrogel-only (collagen hydrogel without a scaffold), and electrospun mesh (ES Mesh) without a hydrogel. Due to rapid confluency and contraction, monolayer and hydrogel only controls were fixed after 4 days. Hydrogel-only controls were seeded with 300 μL of collagen-cell solution and identical cell concentration to composite constructs to ensure a continuous layer covering the entire area of the well in the absence of the scaffold.

Images were acquired with LAS X software using a Leica Thunder (Leica Microsystems) inverted microscope with computational clearing to reduce background noise. Volumetric Z-stack images were acquired at three randomized nonoverlapping locations using the 5× objective (n=3 technical replicates). The DAPI channel was imaged using the 390 nm LED at 40% illumination with a 460 nm emission filter, while the F-actin channel was imaged using the 510 nm LED at 50% illumination with a 535 nm emission filter. All images were acquired with 100 ms exposure time. The maximum intensity projections of the Z-stack were used for analysis. Images of the F-actin channel were pre-processed by converting it to an 8-bit image, enhancing the contrast using histogram equalization, and applying a median filter with a radius of 3.0 pixels. Then a directional analysis based on the Fourier transform was applied to quantify a directional histogram encompassing 180 degrees with 180 discrete bins. The Gaussian distribution contains a single peak, and its goodness-of-fit was used as a uniaxial alignment index. The regional variance of alignment was quantified using the standard deviation of the mean direction of each technical replicate. Images were analyzed and prepared for publication using ImageJ.

Immunostaining and Fluorescence Microscopy: Constructs were fixed with 4% paraformaldehyde (Fisher Scientific) for 30 minutes, permeabilized in 0.1% Triton-X-100 (Millipore Sigma) solution for 20 minutes, blocked in 2% bovine serum albumin (Millipore sigma) for 40 minutes, with phosphate buffered solution (PBS) rinses between each step. Analysis of cellular alignment utilized DAPI (Biolegend) and AlexaFluor 488 phalloidin (Thermo Fisher) to stain nuclei and cellular cytoskeleton. The stains were diluted to 300 nM for DAPI and 400× for phalloidin in blocking solution and incubated for 1 h at room temperature. Analysis of differentiation and maturation utilized a primary antibody to stain myosin heavy chain (MHC) (Abcam, ab91506) with 1:500 dilution incubated in 4 C° overnight. Secondary staining included DAPI, AlexaFluor 568 phalloidin to stain nuclei and cellular cytoskeleton, and 1:200 dilution of AlexaFluor 488 IgG as a secondary antibody (Abcam, ab150081). Samples were incubated in secondary antibodies diluted in blocking solution for 60 minutes at room temperature.

Hydrogel Contraction: The contraction of samples was measured by quantifying the retained scaffold area and comparing it with hydrogel-only controls at day 4 or 8. Constructs were washed with 1× PBS and stained (0.01% toluidine blue) at 0.5 mL/sample for 5 minutes, and then washed again with 1× PBS. A stationary digital camera (Canon) with an ultrashort focal length lens was used to acquire images, which were processed and analyzed in ImageJ. The area was calibrated against standard 24-well dimensions (15.6 mm) and measured for retention percentage compared to the initial area.

Cellular Viability: Cellular viability was assessed 24 h post-seeding using propidium Iodide (PI, ThermoFisher) for dead cells and DAPI for total nuclear count. Z-stack images were acquired at three randomized nonoverlapping locations using the 10× objective (n=3 technical replicates). Constructs with sampled images with an average of less than 30 nuclei per image were excluded from the analysis to avoid inaccurate representation of viability. The images were pre-processed using a median filtered, and the PI channel was thresholded using the positive-control baseline to remove background noise. DAPI images were thresholded manually by visual inspection. The images of each sample were then processed together to compute the Manders' Coefficient (M2), to measure the degree of overlap between the two channels. Viability was defined by V=(1−M2)*100. This ensured the PI signal was specific to a DAPI-positive nuclei. Image data were analyzed using ImageJ and the Coloc2 algorithm.

Cellular Proliferation: Proliferation was measured using the Click-iT® EdU cell proliferation assay (Thermo Fisher). Constructs were incubated with 5-ethynyl-2′-deoxyuridine (EdU) at 10 μM in culture medium at Day 0, fixed after 24 h, stained according to manufacturer guidelines. Briefly, fixed constructs were permeabilized in 0.1% Triton X-100 solution for 20 minutes, then counterstained using anti-EdU reaction solution for 30 minutes at room temperature. Nuclei were counterstained with DAPI, and images were acquired using a Nikon spinning disk confocal microscope with a 10× objective. Volumetric Z-stack images were acquired at three randomized non-overlapping locations, with exclusion criteria of less than 30 total nuclei. Proliferation percentages were quantified based on EdU/DAPI colocalization.

Metabolic Activity and DNA Quantification: Construct metabolic activity (alamarBlue™ assay; Thermo Gisher, A50100) and total DNA content (picoGreen assay; Thermo Fisher, P7589) were assessed 24 h post-seeding. Two cell seeding concentrations were studied, including 400,000 cells/mL and 800,000 cell/mL, comparing composite and scaffold-only constructs while keeping the cell density consistent across the constructs without hydrogel. Isotropic scaffolds were used. Control groups included hydrogel-only without scaffold (Gel-only) and monolayers cultured on standard tissue culture plastic. After 24 h of culture, cells were washed with PBS and incubated in 10% alamarBlue for 2 h. The media was analyzed with a microplate reader with technical duplicates and then averaged. The scaffolds were then digested in 0.5 mg/mL papain (Millipore Sigma, 10108014001) at 65° C. overnight. After 16 hours of incubation, samples were stained with PicoGreen solution, analyzed with a microplate reader, and calibrated against standard dilution curve.

Myoblast differentiation and maturation: Differentiation was evaluated by quantifying myosin heavy chain (MyHC) expression after 16 days, using immunohistochemical staining.

Composite constructs were seeded with cells at a concentration of 800,000 cells/mL, changed to differentiation medium on day 5, and fixed on day 16. Samples were imaged on Zeiss LSM 880 confocal microscope with a 20× objective. The acquisition settings included laser power of 3.0%, 2.4%, and 2.0% for the blue, green, and red excitation lasers, a gain of 849,578, 617.208, and 759.740 for the blue, green, and red emission channels, and a pixel dwell time of 2.576·10⁻⁷ seconds. Images comprised three z stacks per sample at random non-overlapping locations within the constructs, encompassing a depth of the entire sample. The stacks were median filtered using a 3-element cubed structuring element, and maximum intensity projections were obtained. Outcome measures included relative area fraction, myotube count, myotube diameter, and mean fluorescence intensity (MFI). The MFI was acquired from the raw images of the MyHC channel by computing the mean intensity of all pixels. The analysis for the other outcomes followed established protocols from previous publications. The green channel representing MHC expression was processed using localized histogram equalization (CLAHE). The images were then manually thresholded by choosing the triangle criteria as an initial guess and increasing the threshold until the background scatter was fully removed. Then, images were converted to a mask, underwent the morphological imaging operation of opening to remove small objects and filling holes. Myotubes were identified using the Analyze Particles analysis with a lower cutoff of 400 μm. This enabled measurement of the relative area fraction inhabited by myotubes and the number of myotubes within the imagining region. The myotube diameter was then acquired using 5 measurements of the segmented myotubes with no more than 1 measurement per myotube.

Statistical Analyses: The data were analyzed by a one-way ANOVA and a Tukey's post-hoc analysis (95% CI) with the following exception: a two-way ANOVA was used in the hydrogel contraction, nuclear count, cellular viability, proliferation, metabolic activity, and DNA content, to analyze the effects of two independent variables along with Sidak's multiple comparisons tests. Statistical significance was determined at p=0.05, and 0.10>p>0.05 were displayed on graphs. All statistical analyses were conducted using GraphPad Prism version 10.0 (GraphPad Software).

### Example 2

### MEW Enables Fabrication of Large, Aligned, and Stable Scaffolds with Micron-Scale Fibers with Unique Architecture

MEW enabled the fabrication of large scaffolds with 13.0 mm diameter, using high precision 10 μm filament fibers (FIGS. 1A-1I). Each scaffold incorporated two reinforcement rings, fabricated from larger fibers (˜70 μm diameter) along their perimeter, which enhanced handling under wet and dry conditions by resisting folding, bending, and kinking (FIGS. 1A-1C and 3A-3H). SEM confirmed MEW's capacity to manufacture large tissue constructs with micron-sized structural precision (FIGS. 1D-1I). The three scaffold architectures—Isotropic, Aligned T, and Aligned X—demonstrated the versatility of the MEW technology for tailoring the scaffold microenvironments in engineered tissues. The Aligned X architecture featured intermittent diagonal reinforcements (angles of 30-60°), providing superior stability compared to perpendicular reinforcements. Specifically, aligned fibers of the Aligned T scaffold architecture (with perpendicular reinforcements) exhibited deformation between the supports, forming a wavy pattern, while aligned fibers in Aligned X scaffolds maintained their integrity and alignment (FIGS. 4A-4F). Scaffolds with aligned fibers but no reinforcements failed to maintain their shape in aqueous conditions (FIG. 3C). Additionally, while the aligned fibers of Aligned T were interrupted by perpendicular reinforcements (FIGS. 1B, 1E, and 1H), the crossbridge reinforcement in Aligned X enabled highly aligned design features over 10 mm of length without a physical interruption (FIGS. 1C, 1F, and 1I).

### Example 3

### Microfiber Scaffold Architecture Modulates Bulk Mechanical Properties

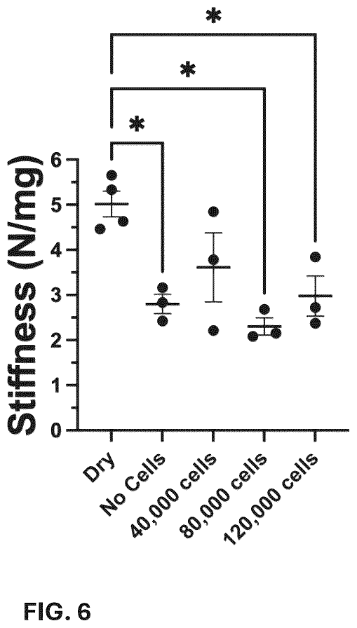

The mechanical properties of microfiber scaffolds were driven by fiber architecture. Tensile testing along the preferred fiber orientation revealed significant differences in the young's modulus, yield stress, and yield strain across the scaffold designs (FIGS. 5A-5C). The Young's modulus had two-fold difference from the lowest to the highest modulus groups, from 3.89±0.575 MPa in the Isotropic group, 6.35±0.800 MPa in the Aligned T, and 10.1±1.62 MPa in the Aligned X group (FIG. 5A), with statistically significant differences between groups (p<0.004). Yield stress of Aligned X and Aligned T scaffolds required significantly more load before plastic deformation, compared with Isotropic scaffolds (p<1·10⁻⁶). Conversely, yield strain indicated the greatest elastic deformation in the Aligned T scaffolds and the lowest in Aligned X (FIG. 5C), with significant differences among all groups (p<0.0350). These results underscore the scaffold architecture's decisive role in modulating bulk mechanical properties. Additionally, scaffold stiffness decreased after incubation with media over 11 days, irrespective of cellular presence (FIG. 6), following a similar observation for MEW scaffolds used as a skin model.

### Example 4

### Aligned Scaffold Architecture Induced Cellular Alignment

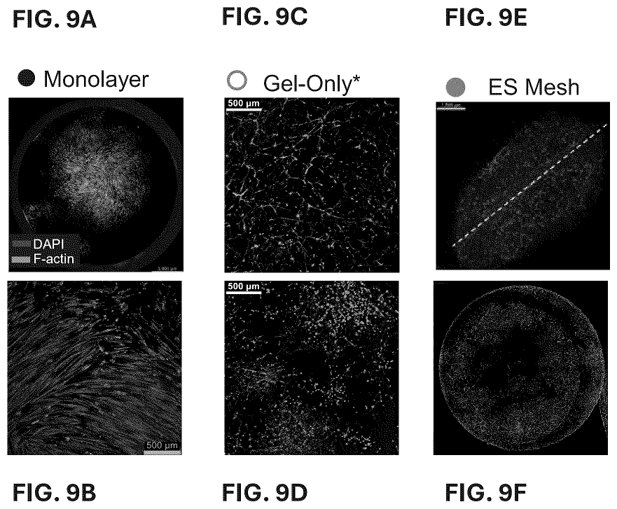

Composite microfiber-hydrogel constructs significantly influenced cellular alignment. By day 8, myoblasts co-localized and aligned closely with the microfiber scaffolds, adopting the scaffold's geometry at both the cellular and tissue scales (FIGS. 7A-7C, 7G-7I, and 8A-8F). Constructs with Aligned T and Aligned X scaffolds resulted in cellular organization with a substantially higher degree of alignment over the isotropic design. The aligned T group had distinct cellular structures along the perpendicular reinforcing fibers (FIGS. 7E and 7H, white arrows). The perpendicular reinforcements (FIG. 1H) impeded formation of continuous cellular bodies across the wall (FIG. 7H). The uniaxial alignment indexes of Aligned T and Aligned X constructs were significantly higher than the Isotropic (p<1·10⁻⁶) and all control groups (p<0.0030, FIG. 7J). The gel-only group had higher alignment index than the Isotropic (p=0.0069) and electrospun constructs (p=0.0092), potentially resulting from the axis of folding due to hydrogel contraction at Day 4 (FIG. 9C). The heterogeneity of cellular organization was measured using the spatial variance of the mean direction. The mean variance of the Aligned X and Aligned T groups were 0.848°±0.509° and 2.908°±3.036° respectively and was significantly lower than 2D monolayer cultures (p<0.0304) and electrospun groups (p<0.0086, FIG. 7K).

### Example 5

### Scaffold Reinforcement Prevents Cell-Mediated Collagen Hydrogel Contraction

Microfiber scaffolds effectively counteracted cell-mediated hydrogel contraction. Collagen hydrogels without reinforcement (FIG. 10A) contracted substantially more by day 4 than composite constructs with Isotropic (FIG. 10B), Aligned T (FIG. 10C), or Aligned X scaffold designs (FIG. 10D). By day 8, hydrogel-only constructs further contracted and assumed an irregular shape (FIG. 10E as compared to FIG. 10F-10H). The amount of hydrogel contraction was attenuated in MEW fiber-reinforced hydrogels, maintaining an average of 87.0%, 84.6%, and 83.64% of area retention on day 4, and 82.7%, 82.7%, and 81.9% of area retention on day 8 for Isotropic, Aligned T, and Aligned X constructs, respectively (FIG. 10I). The contraction of the hydrogel-only constructs differed significantly from day 4 (33.1%) to day 8 (13.2%) with p<0.000001. By contrast, the composite groups mostly maintained their area from day 4 to day 8, with only the Isotropic group statistically decreased in hydrogel area retention from day 4 (87.0%) to day 8 (82.67%) with p=0.0093. The Aligned T group had internal fissures on day 8, with the hydrogel phase separating along the perpendicular reinforcements (FIG. 10G). All constructs had darker toluidine blue staining on day 8, suggesting compositional change in the material (FIGS. 10E-10H). These data demonstrate the microfiber scaffolds reinforced the structural stability of composite engineered muscle constructs by resisting cell-mediated contraction of collagen hydrogel.

### Example 6

### Incorporation of a Hydrogel Enhanced Metabolic Activity and Improved Cellular Seeding Efficiency in Muscle Constructs

The inclusion of hydrogel within composite constructs improved metabolic activity and cell-seeding efficiency. Scaffold-only constructs showed no difference in metabolic activity across the two seeding concentrations (p=0.7708), while composite constructs had increased metabolic activity with higher cell seeding concentration (p=0.0139, FIG. 11A). Analysis of DNA mass revealed consistent results. Composite constructs had increased cell DNA (p=0.0303) with increasing cell seeding density, while the scaffold-only group did not show a difference (p=0.6216, FIG. 11B). Furthermore, a hydrogel-only group with the same levels of cell seeding concentrations revealed increasing metabolic activity and DNA mass with increasing cell density (FIGS. 9A-9F). The high-density composite samples had increased DNA levels compared with the scaffold-only low density (p=0.0410). These results indicate that incorporation of the hydrogel significantly enhanced the number of cells and metabolic activity.

### Example 7

### Incorporation of Collagen Hydrogel Improved Cell Retention and Viability of Composite Constructs Across All Scaffold Designs

The amount of cells retained in muscle constructs and their viability were examined across scaffold-only and composite constructs using nuclear counts and cellular viability. The collagen hydrogel enabled increased number of cells seeded onto constructs with increased viability (FIGS. 12A-12C). A two-way ANOVA showed composite constructs had significantly higher number of nuclei compared with scaffold-only samples (p<0.000001), while the effect of scaffold design was not significant (p=0.3876, FIG. 12A). Scaffold-only constructs of Aligned T and X designs had significantly less nuclei than all composite constructs (FIG. 12A). Composite constructs were consistent with a hydrogel-only group (data not shown), and there were no significant differences between any scaffold designs, suggesting the difference in nuclear count is due to the presence of hydrogel (FIG. 12A). Moreover, cellular viability was significantly increased in composite constructs compared with scaffold-only constructs (p=0.0060), while the scaffold design architecture did not influence viability (p=0.6154, FIG. 12B). The viability of composite constructs was consistent with hydrogel-only controls (data not shown), suggesting the hydrogel enabled increased cellular viability. Still, cells colocalized with and conformed to the scaffold geometry in composite constructs, suggesting the microfiber scaffolds did affect cellular behavior (FIGS. 8A-8F). Furthermore, the proliferation of cells, measured by EdU incorporation, was increased in composite constructs compared with scaffold-only constructs (p=0.0230), and scaffold design also influenced proliferation (p=0.0439, FIG. 12C).

### Example 8

### Composite Constructs with Aligned X Microfiber Scaffold Increased Myotube Formation

Myotube formation and maturation significantly improved in composite constructs with the Aligned X scaffold. Expression of myosin heavy chain (MyHC) protein was compared across Isotropic, Aligned T, and Aligned X composite constructs seeded with C2C12 myoblasts (FIGS. 13A-13J). The Aligned X constructs exhibited larger diameter multinucleated myotubes compared with the Isotropic and Aligned T groups (FIGS. 13A-13F). The area fraction of myotubes in Aligned X constructs were significantly increased from Isotropic (p=0.0151) and nearly significant compared with Aligned T constructs (p=0.0581, FIG. 13G). Together, the data suggest increased expression of MyHC and increased cell differentiation in composite constructs with Aligned X microfiber design. Moreover, the Aligned X constructs also displayed increased maturation. The myotube diameter was significantly higher in the Aligned X group compared with Isotropic (p=0.0007) and Aligned T (p=0.0098) (FIG. 13I). The number of myotubes in Aligned T and X constructs significantly increased compared with the Isotropic group (p=0.0003, and p=0.0007 respectively), while there was no difference between Aligned X and Aligned T (p=0.5157, FIG. 13J). Moreover, the Aligned T myotubes did not cross over perpendicular reinforcements (white arrow, FIG. 13E). Similarly, the Isotropic group had sparse presence of multinucleated structures that were interrupted by reinforcing microfibers.

### Example 9

### In Vivo Evaluation of Microfiber Scaffolds for Treating Volumetric Muscle Loss (VML) Injury

The animal model will be an established tibialis anterior (TA) VML model in 8-12-week-old C57BL/6J mice. A standardized 2×3×2 mm full-thickness defect will be created in the middle of the TA muscle under aseptic surgical conditions while the animal is under anesthesia. The design of the studies and its implementation will be conducted in accordance with IACUC protocols and veterinarian supervision to ensure ethical treatment of all animals.

Mice will be randomized into several treatment groups including (1) microfiber scaffold only, (2) microfiber scaffold with cells (e.g., myoblasts), and (3) microfiber scaffold with collagen hydrogel and cells (e.g., myoblasts); and several control groups including (1) collagen hydrogel only, (2) sham surgery without a defect, and (3) empty defect. The treatment groups with cells will utilize muscle cells from another C57 mice. The cells will start as myoblasts, but over culture, and at the time they are implanted, they will be a heterogenous group including more mature myotubes. The experiment with no cells on the scaffolds prior to implantation can isolate the effect of the scaffold properties on endogenous repair. Primary outcomes of the experiments are functional recovery that includes ankle dorsiflexion torque via peroneal nerve stimulation at 4 and 8 weeks, and histological evaluation at the endpoint. Secondary outcomes include gait and muscle mass restoration. The endpoint of the study will be 8 weeks post injury.

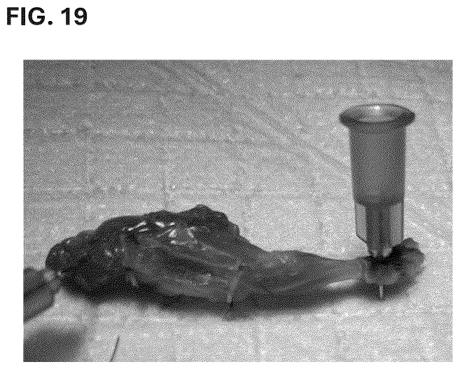

Scaffolds will be sized to fit the defect area and attached using PDO sutures securing the constructs in four corners (FIG. 19). The sutures will ensure the scaffold is fully taut and may impart some small amount of tension. The long axis of the defect will be along a sagittal plane and aligned with the dominant direction of muscle fibers. The direction of aligned scaffold microfibers will be aligned with the long axis of the defect.

We claim:

1. An ultrafine-fiber or microfiber scaffold comprising a plurality of wall fibers and a plurality of reinforcing fibers, wherein each reinforcing fiber comprises at least two anchor segments and at least one bridge segment, wherein:
the wall fibers and the anchor segments are substantially aligned forming a plurality of substantially parallel walls, and the bridge segments each extend across the space between any two adjacent walls from one anchor segment to another of the same reinforcing fiber, forming an angle of less than 90° with the two adjacent walls; and
the reinforcing fibers comprise at least one multi-bridge fiber which connects n adjacent walls with m bridge segments, wherein the m bridge segments are substantially parallel to each other, and n is any integer greater than 2, and m=n−1.

2. The ultrafine-fiber or microfiber scaffold of claim 1, wherein:
the wall fibers constitute about 50.0% to about 99.9% by weight of the scaffold, and the reinforcing fibers constitute about 0.1% to about 50.0% by weight of the scaffold;
the ratio of the number of reinforcing fibers to the number of the wall fibers is about 1:1 to about 1:1,000,000 (reinforcing fibers: wall fibers); and/or
the ratio of the number of reinforcing layers to the number of wall layers is about 1:1 to about 1:1000 (reinforcing layers: wall layers).

3. The ultrafine-fiber or microfiber scaffold of claim 1, wherein the spacing between any two adjacent walls is from about 40 μm to about 500 μm.

4. The ultrafine-fiber or microfiber scaffold of claim 1, wherein the angle formed between the bridge segment and the wall is at least 3°.

5. The ultrafine-fiber or microfiber scaffold of claim 4, wherein the angle formed between the bridge segment and the wall is about 10° to about 45°, about 20° to about 40°, about 25° to about 30°, or about 26° to about 27°.

6. The ultrafine-fiber or microfiber scaffold of claim 1, comprising:
a plurality of repeating multi-bridge fibers each connects n adjacent walls with m bridge segments, wherein the spacing between two adjacent repeating multi-bridge fibers is about x to about (l−m−1)x, wherein l is the number of walls and is any integer greater than 3, and x is the spacing between two adjacent walls.

7. The ultrafine-fiber or microfiber scaffold of claim 1, comprising, for one or more of the multi-bridge fibers, a corresponding mirroring multi-bridge fiber, wherein a bridge segment of the multi-bridge fiber and a bridge segment of the corresponding mirroring multi-bridge fiber pass each other.

8. The ultrafine-fiber or microfiber scaffold of claim 1, wherein the microfiber scaffold has:
a Young's modulus of about 5 MPa to about 15 MPa along the direction of the walls;
a yield point stress of about 450 KPa to about 800 KPa along the direction of the walls; and/or
a yield strain of no more than 20%, along the direction of the walls.

9. The ultrafine-fiber or microfiber scaffold of claim 1, wherein the wall fibers and/or reinforcing fibers comprise poly(α-hydroxy acids), poly(orthoesters), sugars, dextrans, PEG, PEGDA, PCL, PDO, PVA, PLGA, PLCL, PGA, PLA, polyurethanes, nylon, thermoplastic elastomers, liquid crystal elastomers, PEEK, PEKK, or a combination thereof.

10. A composition comprising:
i) the ultrafine-fiber or microfiber scaffold of claim 1, and
ii) a hydrogel, and/or cells.

11. The composition of claim 10, wherein:
the hydrogel comprises type I collagen, gelatin methacrylate, fibrin, agarose, laminin, hyaluronic acid, alginate Matrigel, polyethylene glycol, or any combination thereof; and/or
the cells comprise skeletal muscle cells, ligament cells, tendon cells, cartilage cells, bone cells, vascular cells, fibrogenic cells, neural cells, cardiac cells, or any combination thereof.

12. A method of producing an ultrafine-fiber or microfiber scaffold comprising wall fibers and reinforcing fibers using electrohydrodynamic printing (EHD), comprising:
(a) providing a first set of wall fibers that are substantially parallel to each other; and
(b) EHD jetting along a first straight jetting path that is offset relative to the wall fiber direction at angle of less than 90°, thereby forming a reinforcing fiber.

13. The method of claim 12, wherein
step (a) comprises providing a first set of wall fibers comprising q wall fibers jetted by EHD that are substantially parallel to each other, wherein adjacent wall fibers in the first set are spaced at x, and q is any integer greater than 3; and
step (b) comprises EHD jetting at least one reinforcing fiber along a first diagonal straight jetting path that begins at the same point as a first wall fiber of the set, and ends at the same point as a second wall fiber of the first set, wherein the first wall fiber and second wall fiber are separated by p, wherein p=any value from 3x to (q−1)x.

14. The method of claim 13, further comprising:
repeating step (b) with a second straight jetting path that is parallel to the first straight jetting path, wherein the second and first straight jetting paths are separated by any value from x to (q−p−1)x.

15. The method of claim 13, further comprising:
(c) EHD jetting at least one reinforcing fiber along a first straight mirroring jetting path that mirrors the first straight jetting path.

16. The method of claim 15, further comprising:
repeating step (c) with a second straight mirroring jetting path that is parallel to the first straight mirroring jetting path, wherein the second and first straight mirroring jetting paths are separated by any value from x to (q−p−1)x.

17. The method of claim 15, further comprising before step (c):
EHD jetting a second set of wall fibers having a total of q wall fibers that are substantially parallel to each other and substantially aligned with the wall fibers in the first set, wherein adjacent wall fibers in the second set are spaced at x.

18. The method of claim 13, wherein:
x is from about 40 μm to about 500 μm;
the ratio of the number of reinforcing fibers to the number of wall fibers is from about 1:10 to about 1:1,000,000 (reinforcing fibers:wall fibers); and/or
the wall fibers constitute about 50.0% to about 99.9% by weight of the scaffold, and the reinforcing fibers constitute about 0.1% to about 50.0% by weight of the scaffold.

19. The method of claim 12, wherein the wall fibers and/or reinforcing fibers comprise poly(α-hydroxy acids), poly(orthoesters), sugars, dextrans, PEG, PEGDA, PCL, PDO, PVA, PLGA, PLCL, PGA, PLA, polyurethanes, nylon, thermoplastic elastomers, liquid crystal elastomers, PEEK, PEKK, or a combination thereof.

20. An ultrafine-fiber or microfiber scaffold produced by the method of claim 12.

21. A method of culturing cells, comprising:
culturing cells in the presence of the ultrafine-fiber or microfiber scaffold of claim 1 in a culture medium under conditions sufficient for the cells to grow.

22. A method of treating tissue injury in a subject, comprising:
implanting the ultrafine-fiber or microfiber scaffold of claim 1 at a site of tissue injury in the subject.
